(19) United States
(12) Patent Application Publication
(10) Pub. No.: US 2026/0354671 A1
(43) Pub. Date: Nov. 12, 2026

(54) ENDOSCOPE

(71) Applicant: FUJIFILM Corporation, Tokyo (JP)

(72) Inventors: Ryuji KUWATA, Kanagawa (JP); Takashi HARADA, Kanagawa (JP)

(73) Assignee: FUJIFILM Corporation, Tokyo (JP)

(21) Appl. No.: 19/656,974

(22) Filed: Apr. 23, 2026

(30) Foreign Application Priority Data
May 9, 2025 (JP) 2025-078804

(51) Int. Cl. A61B 1/05; A61B 1/00; A61B 1/005; A61B 1/018; A61B 1/06

(52) U.S. Cl. CPC A61B 1/05; A61B 1/00112; A61B 1/0051; A61B 1/018; A61B 1/0676

(57) ABSTRACT

An endoscope includes: an insertion part to be inserted into a subject; a bendable part disposed in the insertion part and being capable of being bent; a first distal end portion disposed on a distal end side of the bendable part in the insertion part; an illumination portion that is disposed on a distal end surface of the first distal end portion and that emits illumination light; and an observation portion disposed on the distal end surface of the first distal end portion and on which reflected light obtained by reflection of the illumination light by the subject is incident, and the first distal end portion includes a first member in which the observation portion is disposed, a second member adjacent to the first member in a circumferential direction around a longitudinal direction of the insertion part, and an adhesion portion that adheres the first member to the second member.

### CROSS-REFERENCE TO RELATED APPLICATIONS

The present application claims priority under 35 U.S.C. § 119 to Japanese Patent Application No. 2025-078804 filed on May 9, 2025. The above application is hereby expressly incorporated by reference, in its entirety, into the present application.

### BACKGROUND OF THE INVENTION

### 1. Field of the Invention

The disclosed technology relates to an endoscope.

### 2. Description of the Related Art

JP2001-083436A describes an insertion part of an endoscope comprising: a distal rigid portion; an illuminating unit fixed to the distal rigid portion, the illuminating unit of the endoscope including an illumination lens mounted within an illumination lens barrel and a light guide having an emitting end facing the illumination lens; and an observing unit fixed to the distal rigid portion, the observing unit including an objective optical system disposed within an objective lens barrel, in which the distal rigid portion includes a holding portion and a cover portion detachably fitted to the holding portion, recess grooves formed between the holding portion and the cover portion so as to extend radially inward from at least an outer peripheral surface on a holding portion side, the recess grooves forming insertion portions into which the illumination lens barrel and the objective lens barrel are inserted, opening widths of the recess grooves of the holding portion each being equal to or greater than outer diameters of the illumination lens barrel and the objective lens barrel, and a detachable positioning unit provided between the recess grooves and the illumination lens barrel and the objective lens barrel for positioning the illumination lens barrel and the objective lens barrel in an axial direction.

JP2010-119600A describes an endoscope having a distal rigid portion at a distal end portion of an insertion part, in which the distal rigid portion is formed by combining a plurality of units each having a base, the plurality of units being configured such that at least one of an imaging part, an illumination portion, a forceps outlet, or an air/water supply outlet is disposed on at least one of the bases, each base including a curved surface constituting a part of an outer peripheral surface of the distal rigid portion and a surface parallel to an axial direction of the distal rigid portion, and the distal rigid portion is configured such that the surfaces of the bases parallel to the axial direction of the distal rigid portion abut each other to form an overall circular outer periphery.

### SUMMARY OF THE INVENTION

An endoscope according to an aspect of the disclosed technology comprises: an insertion part that is inserted into a subject; a bendable part that is disposed in the insertion part and that is capable of performing a bending operation; a first distal end portion that is disposed on a distal end side of the bendable part in the insertion part; an illumination portion that is disposed on a distal end surface of the first distal end portion and that emits illumination light; and an observation portion that is disposed on the distal end surface of the first distal end portion and on which reflected light obtained by reflection of the illumination light by the subject is incident, in which the first distal end portion includes a first member in which the observation portion is disposed, a second member that is disposed adjacent to the first member in a circumferential direction around a longitudinal direction of the insertion part, and an adhesion portion that adheres the first member to the second member.

### BRIEF DESCRIPTION OF THE DRAWINGS

FIG. 1 is a schematic view showing a schematic configuration of an endoscope device 100 according to an aspect of the disclosed technology.

FIG. 2 is a schematic view showing an internal configuration of a single-use endoscope 40.

FIG. 3 is a perspective view showing a configuration example of a distal end portion 44 and a bendable part 43 of the single-use endoscope 40.

FIG. 4 is a side view showing the configuration example of the distal end portion 44 and the bendable part 43 of the single-use endoscope 40.

FIG. 5 is an exploded perspective view (part 1) of the distal end portion 44 and a first support member 13 shown in FIG. 3.

FIG. 6 is an exploded perspective view (part 2) of the distal end portion 44 and the first support member 13 shown in FIG. 3.

FIG. 7 is a perspective view (part 1) of an insertion part 41 in which a structure 430, the first support member 13, and a coating member 432 are omitted, as viewed from a distal end side.

FIG. 8 is a perspective view (part 2) of the insertion part 41 in which the structure 430, the first support member 13, and the coating member 432 are omitted, as viewed from the distal end side.

FIG. 9 is a front view of the distal end portion 44 shown in FIG. 3 as viewed from the distal end side.

FIG. 10 is an exploded perspective view showing a configuration of an observation module 440.

FIG. 11 is an exploded perspective view (part 1) of a first distal end portion 11.

FIG. 12 is an exploded perspective view (part 2) of the first distal end portion 11.

FIG. 13 is an exploded perspective view (part 3) of the first distal end portion 11.

FIG. 14 is a cross-sectional view showing a butted state between an upper member 111 and a right-side member 112.

FIG. 15 is a cross-sectional view showing a butted state between the upper member 111 and a left-side member 113.

FIG. 16 is a cross-sectional view showing a butted state between the right-side member 112 and the left-side member 113.

FIG. 17 is a schematic view showing an internal configuration of an operation unit 45.

FIG. 18 is an exploded perspective view of a valve mechanism 454.

FIG. 19 is a view showing a state in which a piston 51 is viewed from a direction Y1 side and a direction Y2 side.

FIG. 20 is a schematic cross-sectional view of the valve mechanism 454 in a case where the piston 51 is at a first position P1.

FIG. 21 is a schematic cross-sectional view of the valve mechanism 454 in a case where the piston 51 is at a second position P2.

FIG. 22 is a schematic cross-sectional view of a bending operation unit that performs a bending operation of the bendable part 43 disposed in the operation unit 45.

### DESCRIPTION OF THE PREFERRED EMBODIMENTS

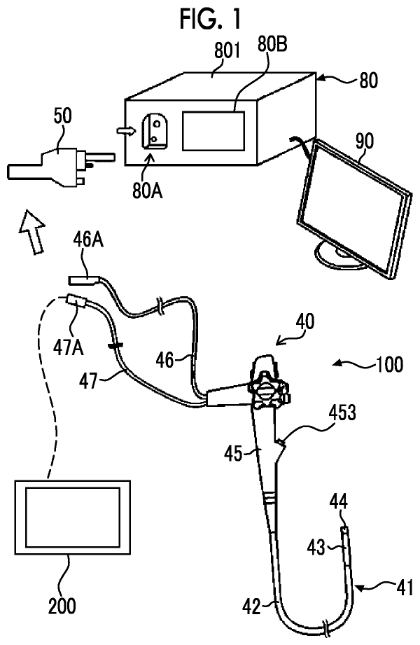

FIG. 1 is a schematic view showing a schematic configuration of an endoscope device 100 according to an aspect of the disclosed technology. As shown in FIG. 1, the endoscope device 100 comprises a control device 80 that can control a reusable endoscope that can be used repeatedly (a plurality of times) by a reprocessing (cleaning and disinfection) treatment, a display device 90 such as a liquid-crystal display or an organic electro-luminescence (EL) display, a single-use endoscope 40 that can be used once (one-time use), and an adapter 50 that relays a connection between the single-use endoscope 40 and the control device 80.

The control device 80 includes an input device 80B (a physical button, a touch panel, a keyboard, a mouse, or the like) that receives an input from a user, and integrally controls the endoscope device 100 in response to an instruction input from the input device 80B.

The control device 80 is connectable to the reusable endoscope and is configured to control the connected reusable endoscope. The control device 80 is connectable to the single-use endoscope 40 via the adapter 50 and is configured to control the single-use endoscope 40 via the adapter 50. The single-use endoscope 40 can be used by being connected to a portable tablet terminal 200 in addition to the control device 80. The single-use endoscope 40 can be controlled by the tablet terminal 200 in a case where a connector 47A is connected to the tablet terminal 200. In addition, the tablet terminal 200 can generate an image and display the image.

The connection between the endoscope and the control device includes at least one of electrical connection between the endoscope and the control device (a state in which transmission and reception of signals or transmission and reception of power can be performed by an electric wire, radio waves, light, or the like), optical connection between the endoscope and the control device (a state in which illumination light can be transmitted and received), or spatial connection between the endoscope and the control device (a state in which transmission and reception of a gas such as air can be performed).

The single-use endoscope 40 is exemplified by a flexible scope, and comprises a flexible insertion part 41 that is inserted into a subject, an operation unit 45 that is disposed on a proximal end side of the insertion part 41 and that is capable of operating the insertion part 41, a first cord part 46 that is provided to extend from the operation unit 45, a connector 46A that is provided at an end portion of the first cord part 46 and that is attachably and detachably connected to the adapter 50, a second cord part 47 that is provided to extend from the operation unit 45, and a connector 47A that is provided at an end portion of the second cord part 47 and that is attachably and detachably connected to the adapter 50. The connector 46A is configured not to be electrically connected to the adapter 50. The connector 47A is configured to be electrically connected to the adapter 50. The connector 46A comprises a connection portion that supplies a medium such as a fluid to the single-use endoscope 40, and is independent of the electrical connection to the adapter 50.

The single-use endoscope 40 is not limited to the flexible scope, and may be another type of endoscope, such as a rigid scope. The single-use endoscope 40 is a gastrointestinal endoscope having a small outer diameter among endoscopes, but is not limited thereto. Each of the first cord part 46 and the second cord part 47 comprises a tubular outer sheath and a member that is inserted and disposed inside the outer sheath.

JP2012-176113A discloses an endoscope comprising an insertion part that includes a bendable part that is bent by a remote operation and a flexible part that is consecutively installed in the bendable part, in which the insertion part has an integrated outer sheath provided over the flexible part and the bendable part, and the outer sheath has a thin-wall portion in at least a part of the bendable part and has a substantially constant outer diameter over the flexible part and the bendable part.

JP1998-66676A (JP-H10-66676A) discloses an endoscope comprising an insertion part that includes a bendable part that is bendable in a predetermined direction, in which the bendable part has a bendable part body that includes at least a curved tube obtained by connecting a plurality of bendable segments, in which an outer sheath tube having an inner diameter greater than an outer diameter of the bendable part body is coated on an outer periphery of the bendable part body in a state of being stretched in a longitudinal direction of the insertion part.

The insertion part 41 includes a bendable part 43 that is capable of performing a bending operation, a distal end portion 44 that is disposed on a distal end side of the bendable part 43, and a flexible part 42 that connects the bendable part 43 and the operation unit 45. The bendable part 43 is formed by connecting a plurality of bending pieces (angle rings), and is configured to bend in a predetermined direction (four directions in the present embodiment) in response to an operation of an angle knob provided in the operation unit 45. The flexible part 42 is elongated and long, and has flexibility.

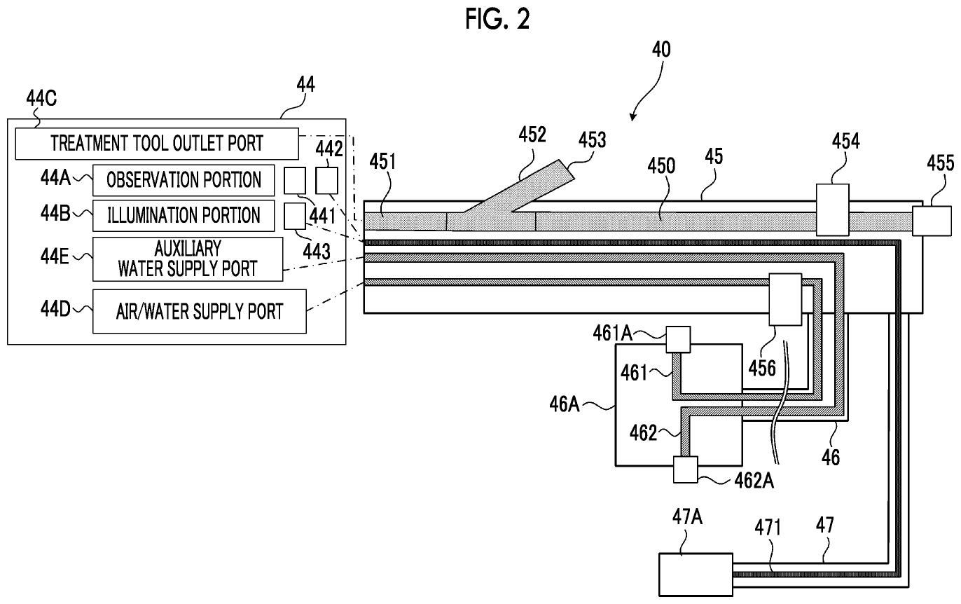

FIG. 2 is a schematic view showing an internal configuration of the single-use endoscope 40. As shown in FIG. 2, a distal end surface of the distal end portion 44 of the single-use endoscope 40 is provided with an illumination portion 44B that emits illumination light, an observation portion 44A on which reflected light obtained by reflection of the illumination light from the illumination portion 44B by a subject is incident, a treatment tool outlet port 44C that is an insertion opening into which a treatment tool is insertable, an air/water supply port 44D, and an auxiliary water supply port 44E.

The air/water supply port 44D is configured to deliver either air (gas) or water (liquid) toward the observation portion 44A. The observation portion 44A can be cleaned by the water delivered from the air/water supply port 44D. Water droplets adhering to the observation portion 44A can be removed by the air delivered from the air/water supply port 44D. The auxiliary water supply port 44E is configured to deliver water (liquid) toward a lumen of the subject, and the water supply makes it possible to remove dirt in the lumen.

The connector 46A of the first cord part 46 is provided with an air/water supply connection fitting 461A to which an air/water supply device can be connected, and an auxiliary water supply fitting 462A to which a syringe or a water supply device that delivers a liquid can be connected.

An air/water supply pipe line 461 as a liquid pipe line and a gas pipe line that communicates with the air/water supply port 44D is provided inside the insertion part 41. The air/water supply pipe line 461 includes, for example, a rigid pipe provided in the distal end portion 44 of the insertion part 41 and a flexible tube connected to the rigid pipe. The air/water supply pipe line 461 is connected to the air/water supply connection fitting 461A of the connector 46A from the air/water supply port 44D through the inside of the insertion part 41, the operation unit 45, and the first cord part 46.

In a case where the air/water supply device including an air supply pump or a water supply tank connected to the air/water supply connection fitting 461A is operated, air or water can be supplied to the air/water supply port 44D through the air/water supply pipe line 461. A valve mechanism 456 is provided inside the operation unit 45 at an intermediate portion of the air/water supply pipe line 461. The valve mechanism 456 is configured as, for example, an air/water supply button. By opening and closing a valve provided in the valve mechanism 456, it is possible to switch on and off the supply of air or water from the air/water supply device connected to the air/water supply connection fitting 461A to the air/water supply port 44D.

A water supply pipe line 462 as a liquid pipe line that communicates with the auxiliary water supply port 44E is provided inside the insertion part 41. The water supply pipe line 462 includes, for example, a rigid pipe provided in the distal end portion 44 of the insertion part 41 and a flexible tube connected to the rigid pipe. The water supply pipe line 462 is connected to the auxiliary water supply fitting 462A of the connector 46A from the auxiliary water supply port 44E through the inside of the insertion part 41, the operation unit 45, and the first cord part 46. In a case where the syringe connected to the auxiliary water supply fitting 462A is operated, water contained in the syringe or water from the water supply device can be supplied to the auxiliary water supply port 44E through the water supply pipe line 462.

A treatment tool pipe line 451 that communicates with the treatment tool outlet port 44C is disposed inside the insertion part 41. The treatment tool pipe line 451 is connected to a branch member 452 inside the operation unit 45. The branch member 452 is a member that branches the treatment tool pipe line 451 into two pipe lines, and an outlet of one of the two pipe lines is a treatment tool inlet port 453 of the operation unit 45.

An operation unit pipe line 450 is connected to an outlet of the other one of the two pipe lines in the branch member 452, and a suction nozzle 455 is connected to the operation unit pipe line 450. The suction nozzle 455 protrudes from a housing of the operation unit 45 and can be connected to a suction device (not shown). A valve mechanism 454 is provided at an intermediate portion of the operation unit pipe line 450. The valve mechanism 454 is configured as, for example, a suction button.

By opening and closing a valve provided in the valve mechanism 454, a negative pressure can be generated by suction of air inside the treatment tool pipe line 451, the branch member 452, and the operation unit pipe line 450 from the suction device connected to the suction nozzle 455. As a result, a body fluid or the like in the subject can be suctioned from the treatment tool outlet port 44C via the treatment tool pipe line 451. As described above, the treatment tool outlet port 44C also functions as a suction port provided on the distal end side of the insertion part 41, and the treatment tool pipe line 451 also functions as a suction pipe line provided to extend from the suction port to the operation unit 45.

The distal end portion 44 of the insertion part 41 is provided with an optical system 441 that forms, as an optical image, reflected light from an observation target site incident on the observation portion 44A, an imaging element 442 that converts the optical image formed by the optical system 441 into an image signal, and a light emitting unit 443 that generates illumination light emitted from the illumination portion 44B. The light emitting unit 443 includes a light emitting element made of a semiconductor device such as a light emitting diode. By using the light emitting element included in the light emitting unit 443 as a semiconductor device, the manufacturing costs of the single-use endoscope 40 can be reduced.

A signal line 471 connected to the imaging element 442 and the light emitting unit 443 is provided inside the insertion part 41. The signal line 471 is connected to the connector 47A from the distal end portion 44 through the inside of the insertion part 41, the operation unit 45, and the second cord part 47. In a case where the connector 47A is connected to the adapter 50, and the adapter 50 is connected to a connector connection portion 80A of the control device 80, the control device 80 and the single-use endoscope 40 are electrically connected to each other. As a result, the control device 80 can control the single-use endoscope 40 and process the imaging signal acquired from the single-use endoscope 40.

In the above description, a configuration in which the adapter 50 and the connector 46A are separable from each other has been described, but the adapter 50 and the connector 46A may be integrated with each other.

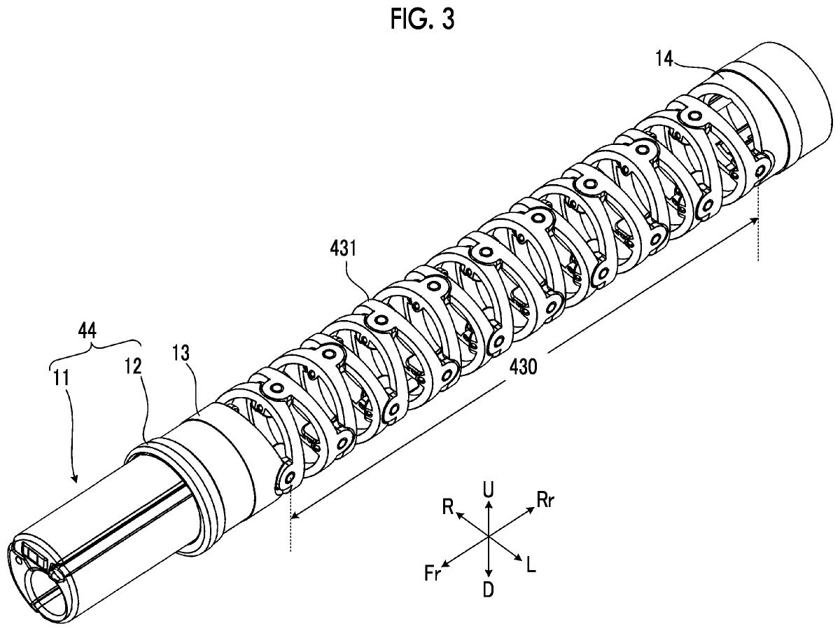

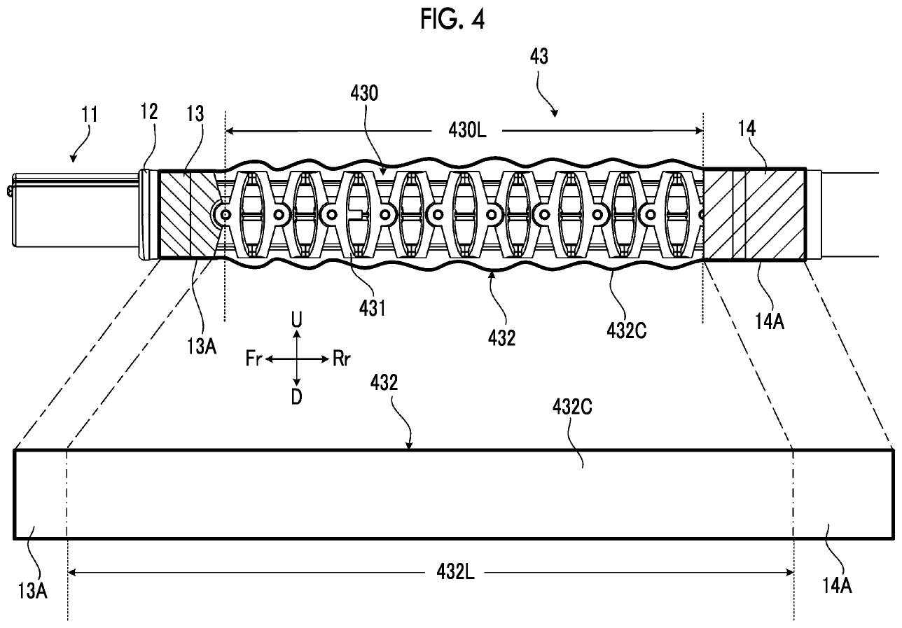

FIG. 3 is a perspective view showing a configuration example of the distal end portion 44 and the bendable part 43 of the single-use endoscope 40. In FIG. 3, a coating member constituting an outer surface of the bendable part 43 is not shown. In addition, structures inserted into the inside of the bendable part 43 are not shown. FIG. 4 is a side view showing the configuration example of the distal end portion 44 and the bendable part 43 of the single-use endoscope 40.

FIG. 3 shows a distal end direction Fr from the proximal end side toward the distal end side and a proximal end direction Rr from the distal end side toward the proximal end side as a longitudinal direction of the insertion part 41 (hereinafter, simply referred to as a longitudinal direction). In addition, FIG. 3 shows an up direction U, a down direction D, a right direction R, and a left direction L among four directions in which a bending operation of the bendable part 43 is possible.

The up direction U and the down direction D are orthogonal to the longitudinal direction (the distal end direction Fr and the proximal end direction Rr) of the insertion part 41. The right direction R and the left direction L are orthogonal to the longitudinal direction of the insertion part 41. The up direction U and the down direction D are orthogonal to the right direction R and the left direction L. The up direction U and the down direction D are collectively referred to as an up-down direction. The left direction L and the right direction R are collectively referred to as a left-right direction.

As shown in FIG. 4, the bendable part 43 comprises a structure 430 that is bendable in four directions by a bending operation and that is formed of a plurality of annular bending pieces 431 arranged in the longitudinal direction and rotatably connected to each other, a tubular first support member 13 that supports a distal end side of the structure 430, a tubular second support member 14 that supports a proximal end side of the structure 430, and a tubular coating member 432 that is made of a non-stretchable material and that serves as an outer sheath of the bendable part 43, the coating member 432 coating the structure 430, the first support member 13, and the second support member 14. The first support member 13 is disposed on a distal end side of the bending piece 431 on the most distal end side of the structure 430, and rotatably supports the bending piece 431. A proximal end side of the second distal end portion 12 is fixed to the first support member 13 on the distal end side thereof.

The second support member 14 is disposed on a proximal end side of the bending piece 431 on the most proximal end side of the structure 430, and rotatably supports the bending piece 431. A distal end side of the flexible part 42 is fixed to the second support member 14 on the proximal end side thereof.

Four operation wires for performing the bending operation of the structure 430 are fixed inside the first support member 13. The four operation wires extend to the inside of the operation unit 45 through the inside of the insertion part 41 and are connected to a pulley to which an angle knob of the operation unit 45 is fixed.

It is preferable that the coating member 432 is made of a non-stretchable material having an elastic modulus of 18 MPa or less at an elongation rate of 100%. The bending piece 431, the first support member 13, and the second support member 14 are made of a resin, so that the manufacturing costs of the single-use endoscope 40 can be reduced. The coating member 432 can be made of, for example, an elastomer, thermoplastic polyurethane, or the like.

As shown in FIG. 3, the distal end portion 44 comprises the first distal end portion 11 and the second distal end portion 12 that is disposed between the first distal end portion 11 and the bendable part 43 and that has an outer diameter greater than that of the first distal end portion 11. A distal end surface of the first distal end portion 11 constitutes a distal end surface of the insertion part 41 or a distal end surface of the distal end portion 44.

As shown in FIG. 4, a distal end side of the coating member 432 is adhered to an outer peripheral surface 13A of the first support member 13 by an adhesive. The outer peripheral surface 13A constitutes a first adhesion portion that adheres the distal end side of the coating member 432 to the first support member 13. A proximal end side of the coating member 432 is adhered to an outer peripheral surface 14A of the second support member 14 by an adhesive. The outer peripheral surface 14A constitutes a second adhesion portion that adheres the proximal end side of the coating member 432 the second support member 14.

The coating member 432 is adhered to the outer peripheral surface 13A and the outer peripheral surface 14A, for example, as follows. An adhesive of an ultraviolet-curable resin is applied to the outer peripheral surface 13A and the outer peripheral surface 14A, and the coating member 432 is fitted over the first support member 13 and the second support member 14. Thereafter, a heat-shrinkable tube is disposed on an outer periphery of the coating member 432 that coats the first support member 13 and the second support member 14. In this state, heat is applied to shrink the heat-shrinkable tube, and the outer peripheral surface 13A and the outer peripheral surface 14A and an inner peripheral surface of the coating member 432 are closely attached to each other. In this state, the adhesive is cured by irradiation with ultraviolet rays.

The coating member 432 is configured such that a first portion 432C between the outer peripheral surface 13A and the outer peripheral surface 14A in the coating member 432 has slack in a state in which the structure 430 extends linearly as shown in FIG. 4. The slack occurs at a plurality of locations in the longitudinal direction. FIG. 4 shows a developed view in which the coating member 432 is stretched in the longitudinal direction such that the slack of the first portion 432C is eliminated.

As shown in the developed view, a length 432L of the first portion 432C in the longitudinal direction is greater than a length 430L of the structure 430 in the longitudinal direction (a distance between a support position of the bending piece 431 on the most distal end side and the first support member 13 and a support position of the bending piece 431 on the most proximal end side and the second support member 14). It is preferable that the length 432L is set such that the first portion 432C has slack even in a state in which the structure 430 is in a bent state (preferably, in a maximally bent state). It is preferable that the length 432L is greater than the length of the structure 430 in the maximally bent state.

The length 432L can be, for example, 1.1 times or more and 1.5 times or less the length 430L. In a case in which the length 432L is smaller than 1.1 times the length 430L, the slack may be reduced in a case in which the structure 430 is bent, and thus resistance to bending may occur. In a case in which the length 432L is greater than 1.5 times the length 430L, the outer diameter of the bendable part 43 increases, which leads to an increase in the diameter of the flexible part 42. Therefore, it is preferable that the length 432L be within the above range.

Although not limited thereto, setting the length 432L to 70 mm or more and 150 mm or less and setting the length 430L to 50 mm or more and 100 mm or less can achieve reduction in the diameter of the insertion part 41 and maintenance of the bending performance. The outer diameter of the first portion 432C can be set to 8 mm or more and 16 mm or less, and the outer diameter of the structure 430 can be set to 9 mm or more and 15 mm or less. By setting these dimensions within the above ranges together with the above-described elastic modulus and elongation rate, deterioration in workability and operability during examination due to slack or tension can be prevented.

The coating member 432 preferably contains a filler. As the filler, for example, a filler having X-ray shielding properties is preferable in order to detect the position of the endoscope from the outside, and, for example, barium sulfate can be employed. The content of the filler in the coating member 432 is preferably set to 51% or less.

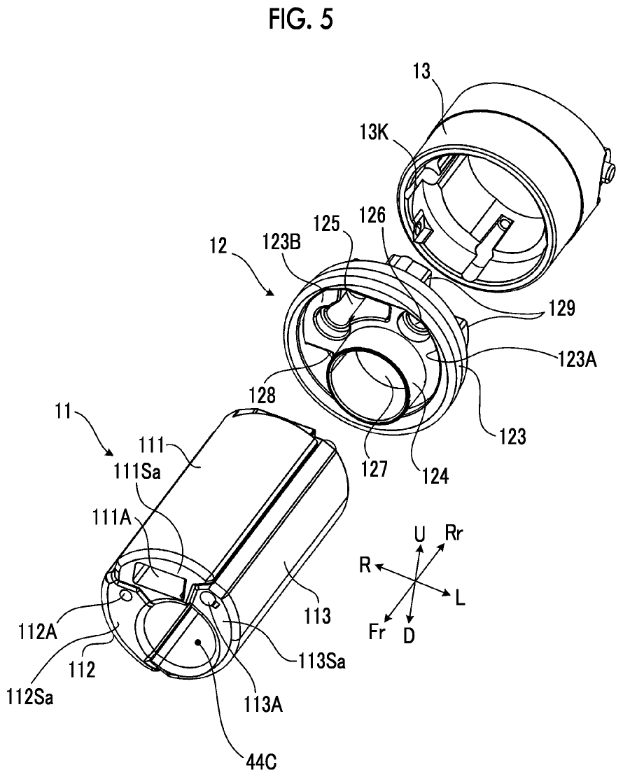

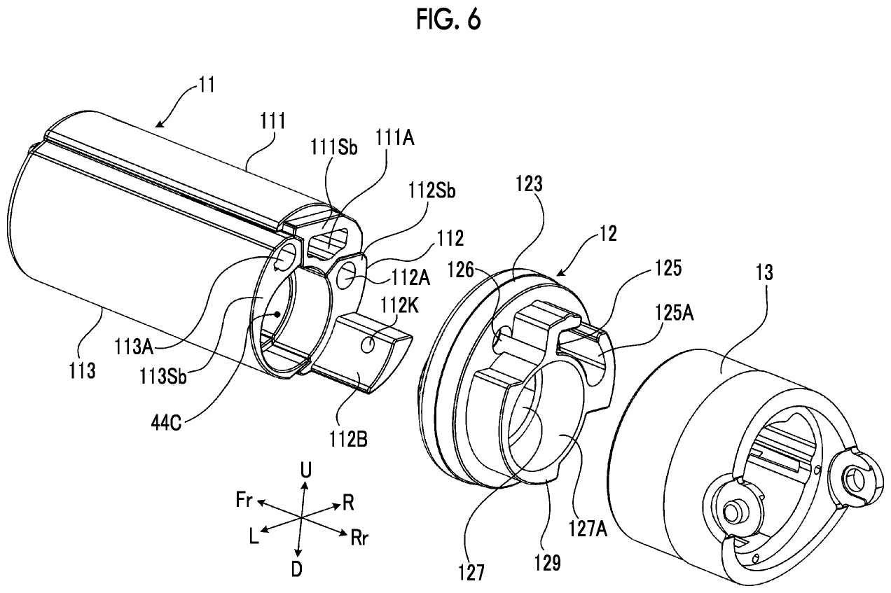

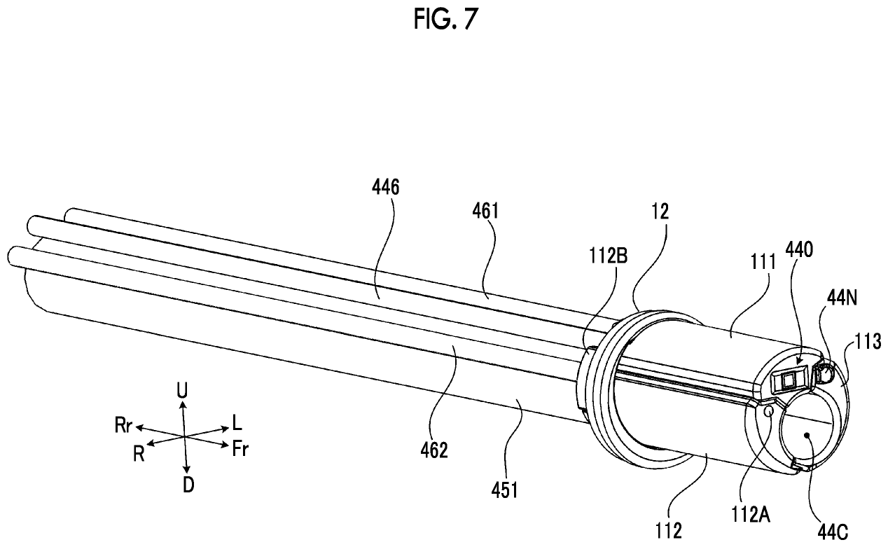

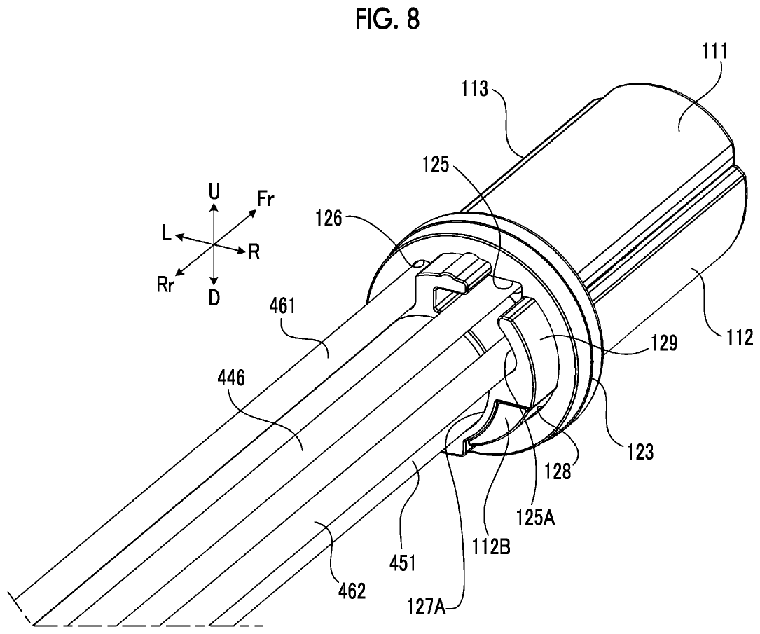

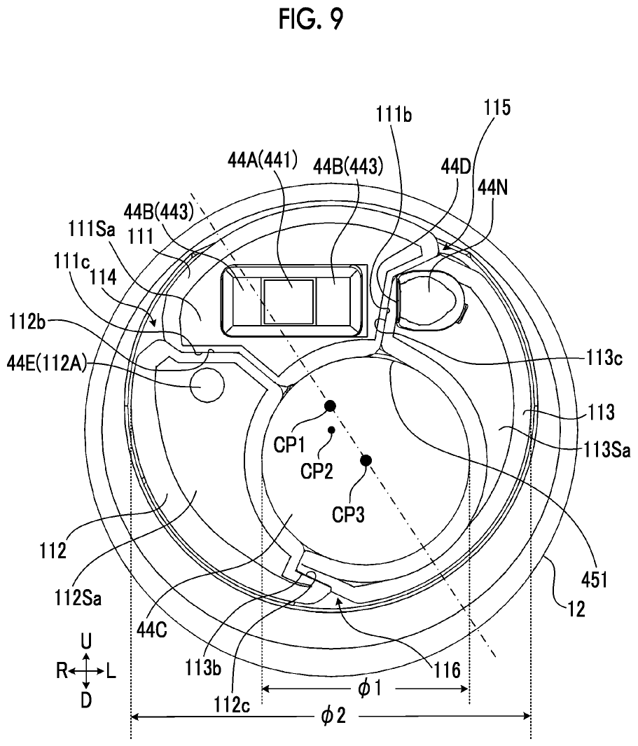

FIG. 5 is an exploded perspective view (part 1) of the distal end portion 44 and the first support member 13 shown in FIG. 3. FIG. 6 is an exploded perspective view (part 2) of the distal end portion 44 and the first support member 13 shown in FIG. 3. FIG. 7 is a perspective view (part 1) of the insertion part 41 in which the structure 430, the first support member 13, and the coating member 432 are omitted, as viewed from the distal end side. FIG. 8 is a perspective view (part 2) of the insertion part 41 in which the structure 430, the first support member 13, and the coating member 432 are omitted, as viewed from the distal end side. FIG. 9 is a front view of the distal end portion 44 shown in FIG. 3 as viewed from the distal end side.

As shown in FIGS. 5 and 6, the second distal end portion 12 has a disk-shaped base portion 123 having a thickness in the longitudinal direction. The base portion 123 is provided with a first through-hole 125, a second through-hole 126, a third through-hole 127, and a fourth through-hole 128 as through-holes that extend in the longitudinal direction. As shown in FIG. 5, a recessed portion 123A is provided on a surface of the base portion 123 on the distal end side. A cylindrical protruding portion 124 that rises toward the distal end side from a peripheral edge of the third through-hole 127 is provided on a bottom surface of the recessed portion 123A.

As shown in FIG. 6, a protruding portion 129 that rises toward the proximal end side from a position inward of an outer peripheral edge of the base portion 123 is provided on a surface of the base portion 123 on the proximal end side. The protruding portion 129 protrudes from a region that does not overlap the second through-hole 126, the third through-hole 127, and the fourth through-hole 128. The protruding portion 129 is inserted into the first support member 13. A cylindrical opening 127A that exposes the third through-hole 127 and an opening 125A that exposes the first through-hole 125 are formed in the protruding portion 129.

As shown in FIG. 8, a distal end side of a cylindrical treatment tool pipe line 451 is inserted into the opening 127A, and the opening 127A and the treatment tool pipe line 451 are fixed to each other by an adhesive or the like. As a result, the treatment tool pipe line 451 and the third through-hole 127 communicate with each other. An inner diameter of the third through-hole 127 and an inner diameter of the treatment tool pipe line 451 match each other.

The treatment tool pipe line 451 includes a cylindrical tube made of a porous resin. The tube is disposed on the innermost side, and an inner diameter thereof is 5.5 mm or more. By including such a tube, the treatment tool pipe line 451 can increase the kink resistance while increasing the inner diameter of the treatment tool pipe line 451. It is preferable that the tube satisfies a kink resistance test of φ20 mm based on JIS T 3215:2021 (or ISO 20697:2018).

In order to satisfy the kink resistance test of φ20 mm while setting the inner diameter of the tube to 5.5 mm or more, it is preferable that a porosity of the tube constituting the treatment tool pipe line 451 be 25% or more. The porosity of the tube can be, for example, 35% or more and 45% or less. In addition, a thickness of the tube constituting the treatment tool pipe line 451 is preferably 0.4 mm or more and 0.8 mm or less. The thickness of the tube can be, for example, 0.6 mm.

As shown in FIG. 2, the treatment tool pipe line 451 is connected to the suction nozzle 455 and also functions as a suction pipe line. Therefore, a viscous outer layer may be provided on an outer periphery of the tube made of a porous resin constituting the treatment tool pipe line 451 over the entire length thereof. As the outer layer, for example, a viscous liquid or semi-solid substance such as grease, wax, or gel can be used. By uniformly providing the outer layer on the surface of the porous tube, the inside of the tube is sealed from the outside even though the tube is porous. Therefore, a sufficient negative pressure can be generated in the treatment tool pipe line 451 during the operation of the valve mechanism 454.

As shown in FIG. 8, a distal end side of a cylindrical air/water supply pipe line 461 is inserted into the second through-hole 126, and the second through-hole 126 and the air/water supply pipe line 461 are fixed to each other by an adhesive or the like.

As shown in FIG. 8, a distal end side of a cylindrical water supply pipe line 462 is inserted into the first through-hole 125 via the opening 125A, and the first through-hole 125 and the water supply pipe line 462 are fixed to each other by an adhesive or the like.

As shown in FIG. 8, a cable 446 in which signal lines 471 are bundled is inserted into the first through-hole 125 via the opening 125A. An observation module 440, which will be described below, is provided at a distal end of the cable 446.

As shown in FIGS. 5 to 9, the first distal end portion 11 includes a plurality of members (three in the illustrated example) arranged in a circumferential direction around the longitudinal direction, and these members are adhered to each other. As shown in FIG. 9, the first distal end portion 11 comprises, as the plurality of members, an upper member 111 disposed on an upper side of a center CP1 of the first distal end portion 11 as viewed in the longitudinal direction, a right-side member 112 disposed on a right side of the center CP1, and a left-side member 113 disposed on a left side of the center CP1. The upper member 111, the right-side member 112, and the left-side member 113 are made of a resin, so that the manufacturing costs can be reduced.

In the circumferential direction of the first distal end portion 11, as shown in FIG. 9, the right-side member 112 is disposed adjacent to the upper member 111 in a counterclockwise direction, the left-side member 113 is disposed adjacent to the right-side member 112 in the counterclockwise direction, and the upper member 111 is disposed adjacent to the left-side member 113 in the counterclockwise direction. The upper member 111 constitutes a first member, one of the right-side member 112 and the left-side member 113 constitutes a second member, and the other of the right-side member 112 and the left-side member 113 constitutes a third member.

In the circumferential direction of the first distal end portion 11, an end surface 111c of the upper member 111 on a right-side member 112 side and an end surface 112b of the right-side member 112 on an upper member 111 side have shapes that allow them to be butted to each other, and they are butted to each other and adhered. The end surface 111c and the end surface 112b are butted to each other via an adhesive made of a resin that is cured by ultraviolet rays, heat, humidity, or the like, and are adhered to each other by curing the adhesive in this state. By using an ultraviolet-curable resin as the adhesive, the manufacturing costs of the first distal end portion 11 can be reduced.

In the circumferential direction of the first distal end portion 11, an end surface 111b of the upper member 111 on a left-side member 113 side and an end surface 113c of the left-side member 113 on the upper member 111 side have shapes that allow them to be butted to each other, and they are butted to each other and adhered. The end surface 111b and the end surface 113c are butted to each other via an adhesive made of a resin that is cured by ultraviolet rays, heat, humidity, or the like, and are adhered to each other by curing the adhesive in this state. By using an ultraviolet-curable resin as the adhesive, the manufacturing costs of the first distal end portion 11 can be reduced.

In the circumferential direction of the first distal end portion 11, an end surface 112c of the right-side member 112 on the left-side member 113 side and an end surface 113b of the left-side member 113 on the right-side member 112 side have shapes that allow them to be butted to each other, and they are butted to each other and adhered. The end surface 112c and the end surface 113b are butted to each other via an adhesive made of a resin that is cured by ultraviolet rays, heat, humidity, or the like, and are adhered to each other by curing the adhesive in this state. The end surface 111b, the end surface 111c, the end surface 112b, the end surface 112c, the end surface 113b, and the end surface 113c constitute butted surfaces. The upper member 111, the right-side member 112, and the left-side member 113 may be temporarily fixed by an adhesive made of an ultraviolet-curable resin, and then finally adhered by curing the adhesive that cures by heat or humidity.

The outer peripheral surface of the first distal end portion 11 has a configuration including partial depressions formed by the shapes of the end portions of the upper member 111, the right-side member 112, and the left-side member 113. For example, a substantially V-shaped recessed portion 114 is formed at a boundary between the upper member 111 and the right-side member 112. A substantially V-shaped recessed portion 115 is formed at a boundary between the upper member 111 and the left-side member 113. A substantially V-shaped recessed portion 116 is formed at a boundary between the right-side member 112 and the left-side member 113. Among the above-described butted surfaces, portions that are exposed on the outer peripheral surface are disposed at the bottoms of the recessed portions 114, 115, and 116. Therefore, even in a case where the cured adhesive provided on the butted surfaces and solidified is exposed to the outer peripheral surface, the adhesive is disposed inside the recessed portion. Therefore, it is possible to prevent the adhesive from coming into contact with the subject. In addition, it is possible to prevent a parting line generated on a mold bonding surface during the manufacturing of the member and a residue thereof from coming into contact with the subject.

In the first distal end portion 11, the manufacturing costs can be reduced by using an ultraviolet-curable resin as the adhesive for adhering the plurality of members. In a case where the ultraviolet-curable resin is used as the adhesive, at least one of the two members to be adhered to each other may be made of a member that transmits ultraviolet light so that the ultraviolet light can reach the butted surfaces of the two members. That is, the butted surfaces of the two members may be configured such that the ultraviolet light transmitted through at least one of the two members can reach the butted surfaces. For example, it is preferable that at least one of the two members has a transmittance of 50% or more with respect to a wavelength of 250 nm or more and 450 nm or less.

As shown in FIGS. 5 and 6, the upper member 111 is provided with a through-hole 111A that extends in the longitudinal direction from an end surface 111Sa on the distal end side to an end surface 111Sb on the proximal end side. The right-side member 112 is provided with a through-hole 112A that extends in the longitudinal direction from an end surface 112Sa on the distal end side to an end surface 112Sb on the proximal end side. The distal end side of the through-hole 112A constitutes an auxiliary water supply port 44E. The left-side member 113 is provided with a through-hole 113A that extends in the longitudinal direction from an end surface 113Sa on the distal end side to an end surface 113Sb on the proximal end side. An air/water supply nozzle 44N shown in FIGS. 7 and 9 is inserted into the through-hole 113A on the distal end side thereof. An outlet of the air/water supply nozzle 44N constitutes the air/water supply port 44D.

As shown in FIG. 5, the through-hole 111A has a substantially rectangular shape extending in the left-right direction. The observation module 440 is disposed inside the distal end side of the through-hole 111A having the substantially rectangular shape.

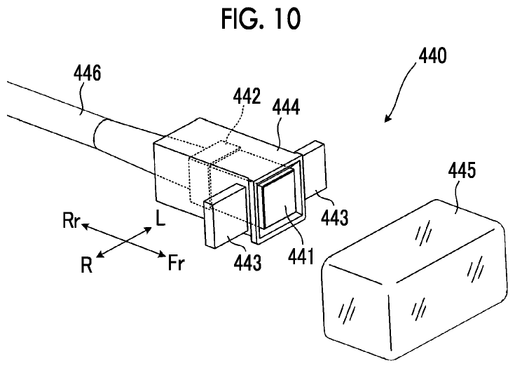

FIG. 10 is an exploded perspective view showing a configuration of the observation module 440. The observation module 440 comprises two light emitting units 443 that are disposed to be spaced apart from each other in the left-right direction, a prism-shaped optical system 441 that is disposed between the two light emitting units 443 and that extends in the longitudinal direction, an imaging element 442 that is disposed on a proximal end side of the optical system 441, a light shielding case 444 that stores the optical system 441 and the imaging element 442, a resin cover 445 that integrally holds the optical system 441, the light emitting unit 443, and the light shielding case 444 and that can transmit visible light, and a cable 446 that includes signal lines 471 connected to the light emitting unit 443 and the imaging element 442. The cable 446 is pulled out from the proximal end side of the through-hole 111A, is inserted into the first through-hole 125 of the second distal end portion 12, and is inserted into the first support member 13 through the opening 125A. The resin cover 445 is formed by molding the distal end side of the observation module 440 with a transparent adhesive.

The light emitting unit 443 comprises a light emitting element such as a light emitting diode (LED) that generates illumination light. The illumination portion 44B includes a region of the resin cover 445 from which light from the light emitting element is emitted.

A region of the resin cover 445 facing the most distal end side of the optical system 441 constitutes an observation portion 44A into which reflected light obtained by reflection of the illumination light from the light emitting unit 443 by the subject is incident. The imaging element 442 is a sensor on which the reflected light from the subject incident on the observation portion 44A forms an image. The observation module 440 is fixed to the through-hole 111A in a state in which the observation portion 44A and the illumination portion 44B face the distal end side.

As shown in FIGS. 5 and 6, the first distal end portion 11 is provided with a treatment tool outlet port 44C formed by a through-hole that extends in the longitudinal direction from a distal end surface on the distal end side to a proximal end surface on the proximal end side. The treatment tool outlet port 44C is formed by a cylindrical space defined by the upper member 111, the right-side member 112, and the left-side member 113. The upper member 111, the right-side member 112, and the left-side member 113 constitute an inner peripheral surface of the treatment tool outlet port 44C.

FIG. 9 shows an inner diameter φ1 of the treatment tool outlet port 44C. The inner diameter φ1 is defined as a diameter of a perfect circle that is set in a case in which the perfect circle is inscribed in a region surrounded by the upper member 111, the right-side member 112, and the left-side member 113. FIG. 9 shows a center CP3 of the treatment tool outlet port 44C that is defined by a center of the perfect circle.

FIG. 9 shows an outer diameter φ2 of the first distal end portion 11. The outer diameter φ2 is defined as a diameter of a perfect circle that is set in a case in which the perfect circle is circumscribed about an outer peripheral edge of the first distal end portion 11. FIG. 9 shows a center CP2 of the second distal end portion 12.

In the single-use endoscope 40, a ratio of the inner diameter φ1 of the treatment tool outlet port 44C to the outer diameter φ2 of the first distal end portion 11 is 0.37 or more. By setting the ratio to 0.37 or more, it is possible to reduce the diameter of the insertion part 41 and to improve the treatment efficiency using the treatment tool. By setting the ratio to 0.37 or more, it is also possible to use a large treatment tool, and it is possible to expand the range of treatments. In addition, it is possible to increase a suction force for blood or the like using the treatment tool outlet port 44C. With these effects, it is possible to enhance the value of the single-use endoscope 40.

In a case in which it is desired to increase the durability and the cleanability of the single-use endoscope 40, it is difficult to set the ratio to 0.37 or more. However, since the single-use endoscope 40 is for single use, the ratio can be set to 0.37 or more by setting the durability and the cleanability to minimum necessary levels.

In consideration of the manufacturing suitability of the first distal end portion 11, it is preferable that the outer diameter φ2 be 11.6 mm or more and the inner diameter φ1 be 4.3 mm or more. As a result of various verifications, it has been found that the single-use endoscope 40 in which the outer diameter φ2 is 11.6 mm, the inner diameter φ1 is 6 mm, and the ratio is approximately 0.51 can be manufactured.

As shown in FIG. 9, the air/water supply port 44D, which is a fluid delivery outlet of the air/water supply nozzle 44N, is disposed toward two illumination portions 44B and an observation portion 44A, which are arranged in a straight line in the left-right direction. In other words, the air/water supply port 44D of the air/water supply nozzle 44N is disposed on an extension line of a line segment connecting the observation portion 44A and the illumination portion 44B. In addition, a straight line extending from the air/water supply port 44D of the air/water supply nozzle 44N in a delivery direction of the fluid does not intersect the treatment tool outlet port 44C.

With such a configuration, air or liquid delivered from the air/water supply nozzle 44N can be efficiently directed toward the observation portion 44A and the illumination portion 44B, and the cleaning effect of the observation portion 44A and the illumination portion 44B can be enhanced. In addition, since the treatment tool outlet port 44C is not provided in a flow path of air or liquid delivered from the air/water supply nozzle 44N, the use of the treatment tool is not affected.

By setting the ratio to 0.37 or more, the following characteristic configuration can be obtained in a front view as viewed along the longitudinal direction.

As shown in FIG. 9, in a first direction passing through the center CP1 of the first distal end portion 11 and the center CP3 of the treatment tool outlet port 44C (a direction in which a one-dot chain line shown in the drawing extends), the observation portion 44A and the air/water supply nozzle 44N are positioned on a center CP1 side with respect to the center CP3 (positioned closer to the center CP1 than to the center CP3).

The treatment tool outlet port 44C is disposed in a region overlapping the center CP1 of the first distal end portion 11.

In the first direction, the air/water supply nozzle 44N is positioned on the center CP1 side of the first distal end portion 11 with respect to the observation portion 44A.

In a second direction perpendicular to the first direction, the observation portion 44A and the air/water supply nozzle 44N are disposed at different positions.

The illumination portion 44B is positioned on the center CP1 side of the first distal end portion 11 with respect to the center CP3 of the treatment tool outlet port 44C (positioned closer to the center CP1 than to the center CP3).

The center CP1 of the first distal end portion 11 and the center CP2 of the second distal end portion 12 are at different positions. In addition, the treatment tool outlet port 44C is disposed in a region including the center CP1 of the first distal end portion 11 and the center CP2 of the second distal end portion 12. With such a disposition, the bending of the treatment tool pipe line 451 is suppressed, and the operability of the treatment tool is maintained.

As shown in FIG. 5, the proximal end side of the first distal end portion 11 is inserted into the recessed portion 123A of the second distal end portion 12, and the outer peripheral surface thereof is held by the second distal end portion 12. In this way, the upper member 111, the right-side member 112, and the left-side member 113 are disposed on the inside of the second distal end portion 12 on the proximal end side thereof and are held by the second distal end portion 12, so that the upper member 111, the right-side member 112, and the left-side member 113 can be prevented from being separated outward in a radial direction of the first distal end portion 11.

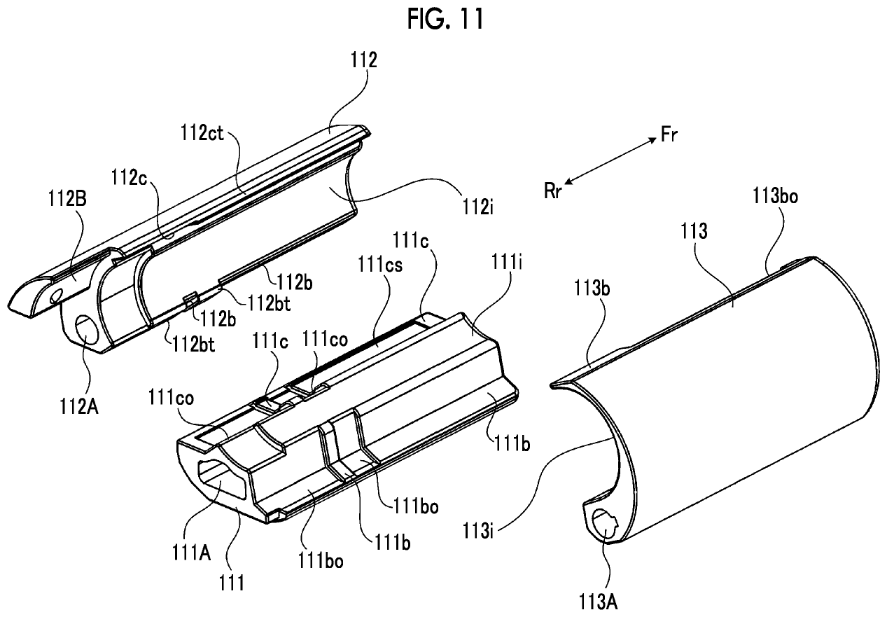

The protruding portion 124 of the second distal end portion 12 is inserted into the treatment tool outlet port 44C of the first distal end portion 11 on the proximal end side, so that the treatment tool pipe line 451 inserted into the opening 127A and the treatment tool outlet port 44C communicate with each other. In addition, the first through-hole 125 of the second distal end portion 12 and the through-hole 111A and the through-hole 112A of the first distal end portion 11 communicate with each other, and the second through-hole 126 of the second distal end portion 12 and the through-hole 113A of the first distal end portion 11 communicate with each other. As a result, the water supply pipe line 462 inserted into the first through-hole 125 and the through-hole 112A communicate with each other, and the air/water supply pipe line 461 inserted into the second through-hole 126 and the air/water supply nozzle 44N communicate with each other. The through-hole 113A has a keyhole shape into which a fixing projection portion (not shown) provided on the air/water supply nozzle 44N is fitted, and the keyhole shape is formed over the entirety of the through-hole 113A (see FIG. 11).

As shown in FIG. 6, an extending portion 112B extending to the proximal end side is provided on an end surface 112Sb of the right-side member 112. As shown in FIG. 8, the extending portion 112B is inserted into the fourth through-hole 128 of the second distal end portion 12 and is positioned on the proximal end side with respect to the base portion 123. A portion extending to the proximal end side with respect to the base portion 123 is inserted into the first support member 13, which is a tubular member. As shown in FIG. 5, a through-hole 13K penetrating in a radial direction of the first support member 13 is formed in a region facing the extending portion 112B in the first support member 13. The extending portion 112B is provided with a hole portion 112K formed by a through-hole extending in the same direction as the through-hole 13K at a position facing the through-hole 13K. The through-hole 13K and the hole portion 112K are filled with an adhesive such as an ultraviolet-curable resin and cured. The adhesive serves as a fixing pin between the first distal end portion 11 and the first support member 13, so that the position of the first distal end portion 11 in the longitudinal direction is firmly maintained.

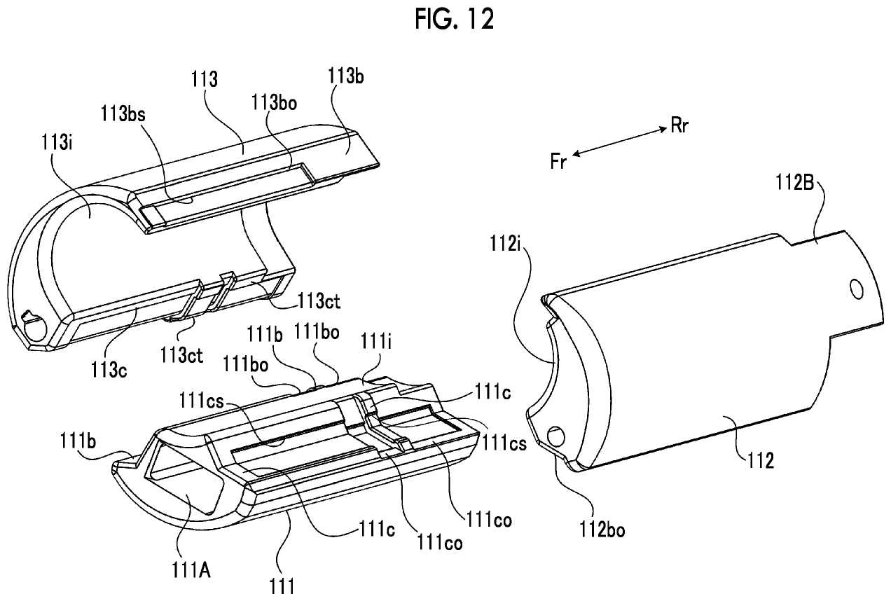

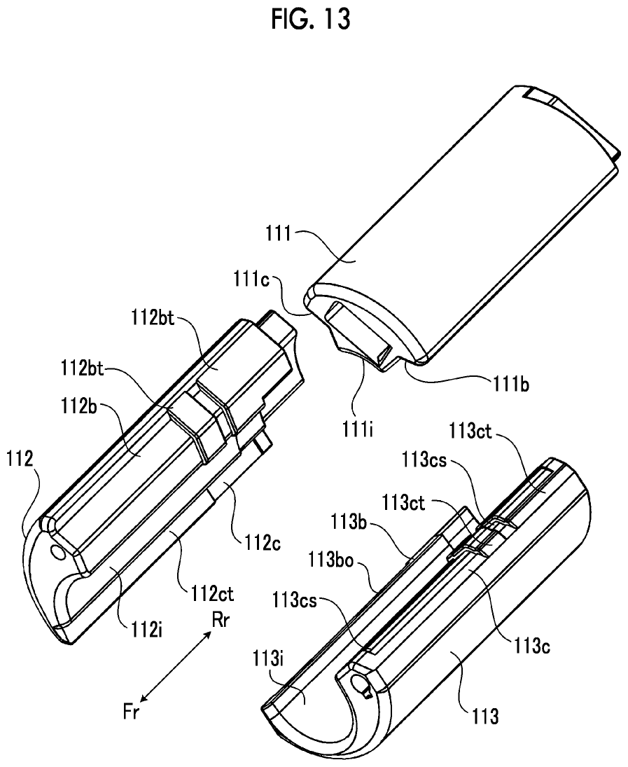

FIG. 11 is an exploded perspective view (part 1) of the first distal end portion 11. FIG. 12 is an exploded perspective view (part 2) of the first distal end portion 11. FIG. 13 is an exploded perspective view (part 3) of the first distal end portion 11.

As shown in FIGS. 11 and 12, the upper member 111 comprises an arc-shaped curved surface 111i that forms an inner peripheral surface of the treatment tool outlet port 44C, an end surface 111c having a V-shaped cross section that connects one end of the curved surface 111i on the right-side member 112 side in the circumferential direction and an outer peripheral surface of the upper member 111, and an end surface 111b having a V-shaped cross section that connects the other end of the curved surface 111i on the left-side member 113 side in the circumferential direction and the outer peripheral surface of the upper member 111.

As shown in FIG. 11, two recessed portions 111bo are formed on the end surface 111b to be spaced apart from each other in the longitudinal direction. As shown in FIG. 12, two recessed portions 111co are formed on the end surface 111c to be spaced apart from each other in the longitudinal direction. In addition, an adhesive reservoir 111cs formed by a recessed portion to which an adhesive is applied is formed on the end surface 111c to extend across the recessed portions 111co.

As shown in FIGS. 12 and 13, the left-side member 113 comprises an arc-shaped curved surface 113i that forms an inner peripheral surface of the treatment tool outlet port 44C, and an end surface 113c having a V-shaped cross section that connects one end of the curved surface 113i on the upper member 111 side in the circumferential direction and an outer peripheral surface of the left-side member 113. Two projection portions 113ct are formed on the end surface 113c to be spaced apart from each other in the longitudinal direction. An adhesive reservoir 113cs formed by a recessed portion to which an adhesive is applied is formed on the end surface 113c to extend across the projection portions 113ct.

As shown in FIG. 13, the right-side member 112 comprises an arc-shaped curved surface 112i that forms an inner peripheral surface of the treatment tool outlet port 44C, an end surface 112b having a V-shaped cross section that connects one end of the curved surface 112i on the upper member 111 side in the circumferential direction and an outer peripheral surface of the right-side member 112, and an end surface 112c having a V-shaped cross section that connects the other end of the curved surface 112i on the left-side member 113 side in the circumferential direction and the outer peripheral surface of the right-side member 112. A projection portion 112ct is formed on the end surface 112c. Two projection portions 112bt are formed on the end surface 112b to be spaced apart from each other in the longitudinal direction.

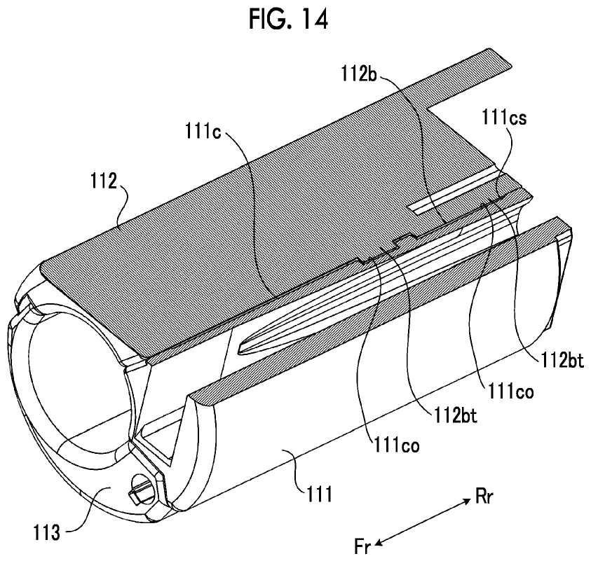

FIG. 14 is a cross-sectional view showing a butted state between the upper member 111 and the right-side member 112. In a state in which the adhesive is applied to the adhesive reservoir 111cs, the end surface 111c of the upper member 111 and the end surface 112b of the right-side member 112 are butted to each other. In a state in which the end surface 111c and the end surface 112b are butted to each other, the projection portion 112bt of the right-side member 112 engages with the recessed portion 111co of the upper member 111. In this state, the adhesive is cured by the ultraviolet light reaching the adhesive reservoir 111cs, so that the upper member 111 and the right-side member 112 are adhered to each other by a region (constituting a second adhesion region) of the adhesive reservoir 111cs and the end surface 112b of the right-side member 112 facing the adhesive reservoir 111cs. In addition, the upper member 111 and the right-side member 112 are restricted from moving relative to each other in the longitudinal direction by the engagement between the recessed portion 111co and the projection portion 112bt, and are adhered to each other in the longitudinal direction. The recessed portion 111co and the projection portion 112bt constitute a first adhesion region or a restricting structure.

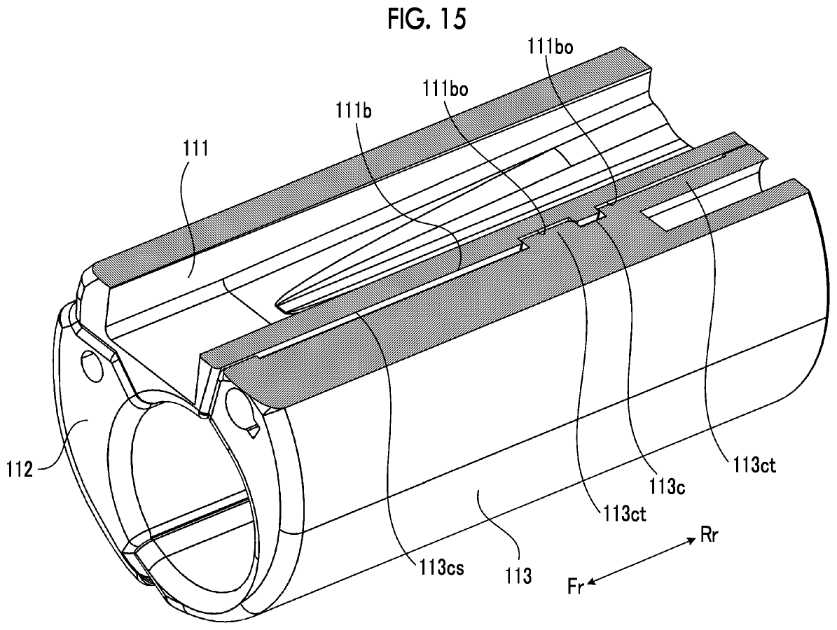

FIG. 15 is a cross-sectional view showing a butted state between the upper member 111 and the left-side member 113. In a state in which the adhesive is applied to the adhesive reservoir 113cs, the end surface 111b of the upper member 111 and the end surface 113c of the left-side member 113 are butted to each other. In a state in which the end surface 111b and the end surface 113c are butted to each other, the projection portion 113ct of the left-side member 113 engages with the recessed portion 111bo of the upper member 111. In this state, the adhesive is cured by the ultraviolet light reaching the adhesive reservoir 113cs, so that the upper member 111 and the left-side member 113 are adhered to each other by a region (constituting a second adhesion region) of the adhesive reservoir 113cs and the end surface 111b of the upper member 111 facing the adhesive reservoir 113cs. In addition, the upper member 111 and the left-side member 113 are restricted from moving relative to each other in the longitudinal direction by the engagement between the recessed portion 111bo and the projection portion 113ct, and are adhered to each other in the longitudinal direction. The recessed portion 111bo and the projection portion 113ct constitute a first adhesion region or a restricting structure.

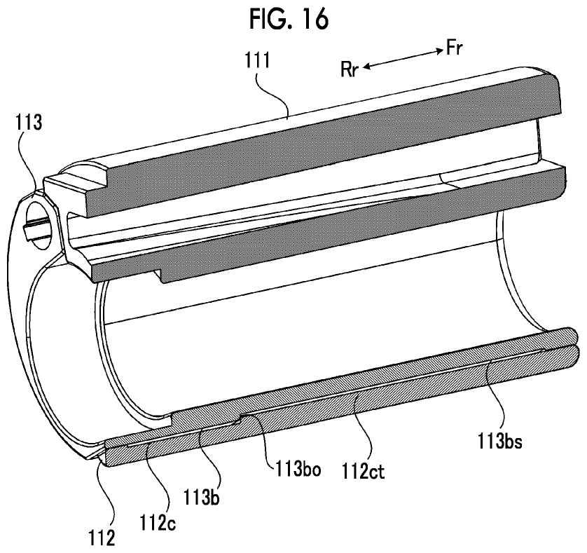

FIG. 16 is a cross-sectional view showing an abutted state between the right-side member 112 and the left-side member 113. In a state in which the adhesive is applied to the adhesive reservoir 113bs, the end surface 113b of the left-side member 113 and the end surface 112c of the right-side member 112 are butted to each other. In a state in which the end surface 113b and the end surface 112c are butted to each other, the projection portion 112ct of the right-side member 112 engages with the recessed portion 113bo of the left-side member 113. In this state, the adhesive is cured by the ultraviolet light reaching the adhesive reservoir 113bs, so that the right-side member 112 and the left-side member 113 are adhered to each other by a region (constituting a second adhesion region) of the adhesive reservoir 113bs and the end surface 112c of the right-side member 112 facing the adhesive reservoir 113bs. In addition, the right-side member 112 and the left-side member 113 are restricted from moving relative to each other in the longitudinal direction by the engagement between the recessed portion 113bo and the projection portion 112ct, and are adhered to each other in the longitudinal direction. The recessed portion 113bo and the projection portion 112ct constitute a first adhesion region or a restricting structure.

As described above, the upper member 111, the right-side member 112, and the left-side member 113 are adhered to each other by an adhesion portion including the first adhesion region and the second adhesion region having different shapes. The first adhesion region and the second adhesion region are positioned inward of the outer peripheral surface of the first distal end portion 11 and are not exposed. Therefore, the adhesive does not come into contact with the subject, and the parting line generated on the mold bonding surface during the manufacturing of the member and the residue thereof do not come into contact with the subject, so that safety can be improved.

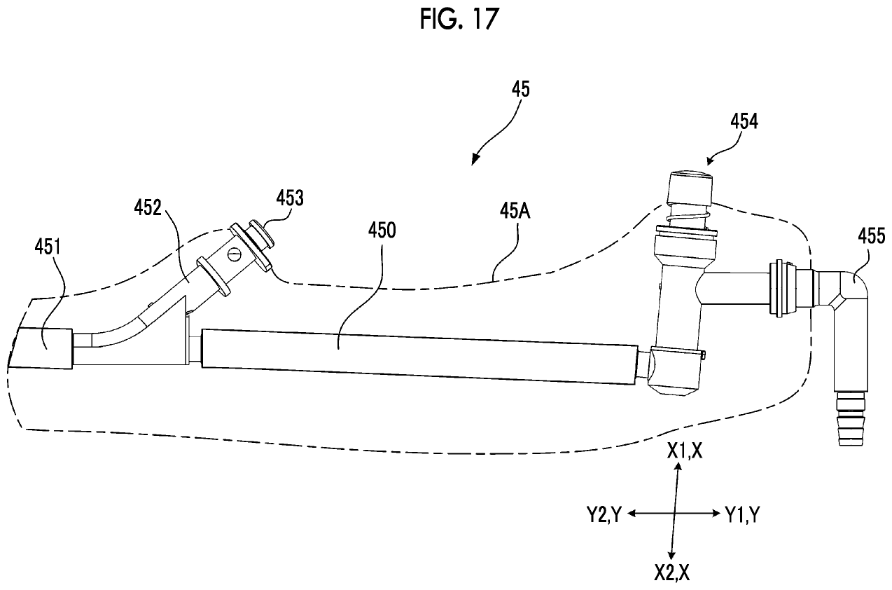

FIG. 17 is a schematic view showing an internal configuration of the operation unit 45. Inside a housing 45A of the operation unit 45 are provided a branch member 452 that is connected to a proximal end of a treatment tool pipe line 451 and that branches the treatment tool pipe line 451 into a first pipe line on a treatment tool inlet port 453 side and a second pipe line on a suction nozzle 455 side, an operation unit pipe line 450 of which a distal end side is connected to an outlet of the second pipe line of the branch member 452, a valve mechanism 454 that is connected to a proximal end side of the operation unit pipe line 450, and a suction nozzle 455 that is connected to the valve mechanism 454. Since the operation unit pipe line 450 communicates with the treatment tool pipe line 451 via the branch member 452, the operation unit pipe line 450 constitutes a pipe line that is disposed in the operation unit 45 and that communicates with the treatment tool outlet port 44C.

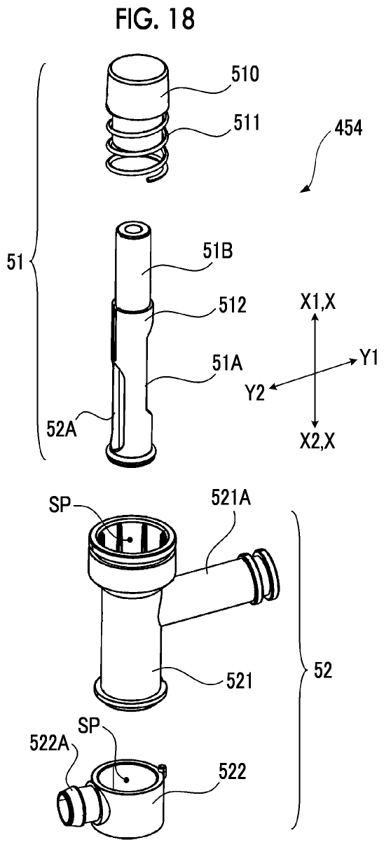

FIG. 18 is an exploded perspective view of the valve mechanism 454. The valve mechanism 454 is configured as a suction button that is operable by being pressed in a direction X2, and comprises a piston 51 that is an operation member which is movable and has an internal space and that performs an operation of causing the operation unit pipe line 450 and the suction nozzle 455 to communicate with each other, and a storage member 52 that forms a storage space SP in which the piston 51 is movably stored, and that has a first connection pipe line 522A that connects the operation unit pipe line 450 and the storage space SP to each other and a second connection pipe line 521A that connects the suction nozzle 455 and the storage space SP to each other.

In FIG. 18, a direction opposite to the direction X2 is shown as a direction X1. The direction X1 and the direction X2 are collectively referred to as a direction X. The direction X is a direction in which the piston 51 is movable. In FIG. 18, a direction Y intersecting the direction X is shown. In the direction Y, a direction from the operation unit pipe line 450 toward the suction nozzle 455 is shown as a direction Y1, and a direction opposite to the direction Y1 is shown as a direction Y2.

The piston 51 comprises a tubular body 512, a button 510 that is consecutively installed in the tubular body 512 and that constitutes a protruding portion protruding from the housing 45A, and a spring 511 that is fixed to the button 510 and the storage member 52 and that biases the button 510 in the direction X1. Both ends of the tubular body 512 are closed, and the button 510 is externally fitted and fixed to a columnar end portion 51B on a direction X1 side.

Hole portions 51A and 52A that do not allow the first connection pipe line 522A and the second connection pipe line 521A to communicate with each other in a case where the piston 51 is at a first position P1 in the storage space SP and that allow the first connection pipe line 522A and the second connection pipe line 521A to communicate with each other in a case where the piston 51 is at a second position P2 in the storage space SP are formed in an outer peripheral surface of the tubular body 512. The first position P1 is a position of the piston 51 in a state in which the button 510 is not pressed in the direction X2. The second position P2 is a position of the piston 51 in a state in which the button 510 is pressed in the direction X2.

The hole portion 52A is formed in an outer peripheral surface of the tubular body 512 on a direction Y2 side and is configured as an opening that communicates with an internal space of the tubular body 512. The hole portion 51A is formed in an outer peripheral surface of the tubular body 512 on a direction Y1 side and is configured as an opening that communicates with the internal space of the tubular body 512. The hole portion 52A constitutes a first opening, and the hole portion 51A constitutes a second opening.

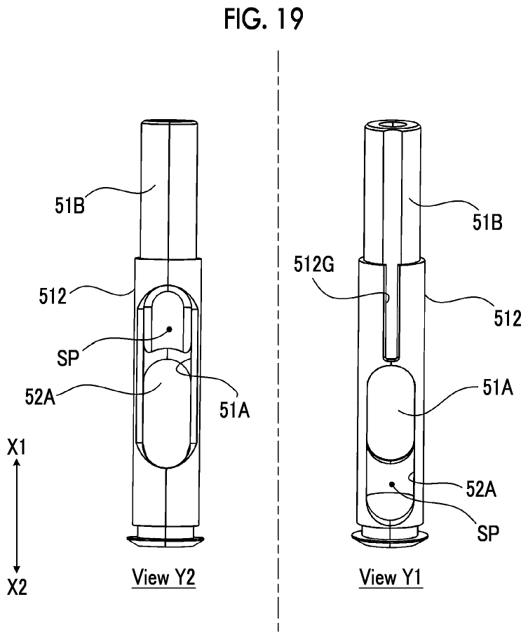

FIG. 19 is a view showing a state in which the piston 51 is viewed from the direction Y1 side and the direction Y2 side. As shown in FIG. 19, the hole portion 52A and the hole portion 51A are each configured as an elongated hole extending in the direction X. The hole portion 52A and the hole portion 51A are at different positions in the direction X, and the hole portion 52A is disposed on a direction X2 side with respect to the hole portion 51A. A guide groove 512G extending in the direction X is formed on the outer peripheral surface of the tubular body 512 adjacent to the hole portion 52A on the direction X1 side.

The storage member 52 includes a bottomed tubular first storage member 522 including a first connection pipe line 522A and a tubular second storage member 521 including a second connection pipe line 521A, the first storage member 522 and the second storage member 521 being connected to each other.

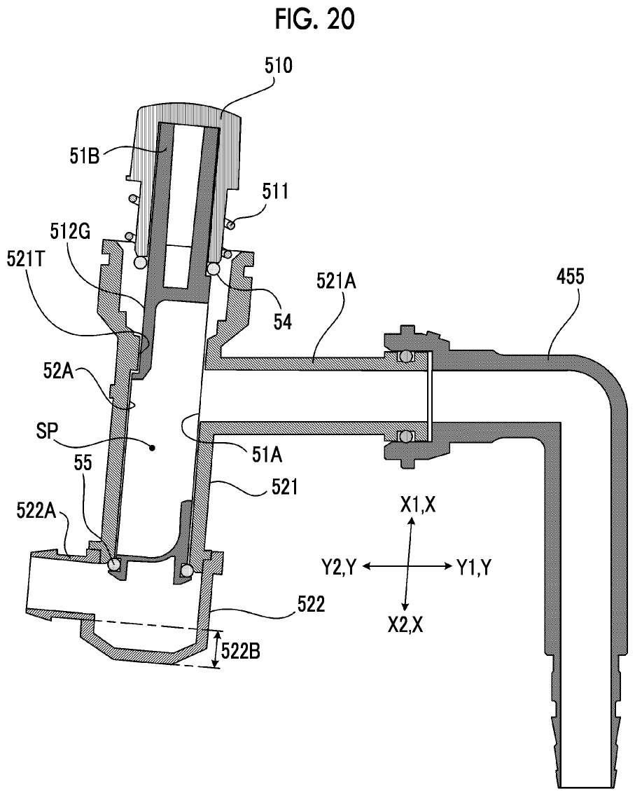

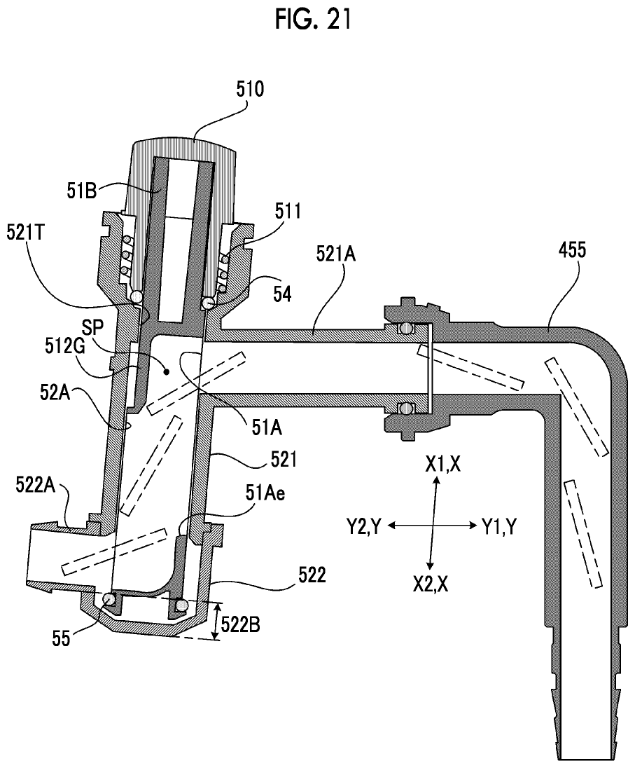

FIG. 20 is a schematic cross-sectional view of the valve mechanism 454 in a case where the piston 51 is at the first position P1. FIG. 21 is a schematic cross-sectional view of the valve mechanism 454 in a case where the piston 51 is at the second position P2. As shown in FIGS. 20 and 21, an end portion of the second storage member 521 on the direction X2 side is inserted into the first storage member 522. The first connection pipe line 522A is provided to protrude in the direction Y2 from a side surface of the first storage member 522. The second connection pipe line 521A is provided to protrude in the direction Y1 from a side surface of the second storage member 521. An L-shaped suction nozzle 455 is externally fitted in a rotatable manner to an end portion of the second connection pipe line 521A.

An end portion of the second storage member 521 on the direction X1 side has an increased diameter so as to provide a gap with the piston 51. An engaging projection portion 521T that engages with the guide groove 512G of the piston 51 is provided on an inner peripheral surface of the second storage member 521. The guide groove 512G of the piston 51 and the engaging projection portion 521T of the storage member 52 engage with each other, so that the rotation of the piston 51 in the storage member 52 is restricted.

In a case where the piston 51 moves in the direction X, the relative movement of the engaging projection portion 521T with respect to the piston 51 is guided by the guide groove 512G, so that the piston 51 can be moved smoothly. In a state shown in FIG. 20 in which the button 510 is not pressed, an end edge of the guide groove 512G on the direction X2 side comes into contact with the engaging projection portion 521T, so that the movement of the piston 51 in the direction X1 is restricted. Accordingly, the piston 51 can be prevented from coming out of the storage member 52. In assembly, the tubular body 512 is inserted from an opening of the second storage member 521 on a first storage member 522 side in a state in which the first storage member 522 is removed. In this case, the engaging projection portion 521T is guided by the guide groove 512G. Thereafter, an O-ring 54 and the spring 511 are mounted on the end portion 51B protruding from the second storage member 521, and the button 510 is attached. Further, the first storage member 522 is attached to the second storage member 521.

The first storage member 522 has a bottom portion 522B as a portion positioned on a side opposite to a second connection pipe line 521A side with respect to the first connection pipe line 522A in the direction X. As shown in FIG. 21, in a case where the piston 51 is at the second position P2, an end portion of the piston 51 on the direction X2 side is stored in the bottom portion 522B. An O-ring 55 as a sealing member is provided on an outer periphery of the end portion of the piston 51. The piston 51 is provided with the O-ring 54 as a sealing member on an outer periphery of the end portion 51B and adjacent to an end surface of the button 510 on the direction X2 side. The button 510 is configured in a tubular shape having a large-diameter portion on the direction X1 side and a small-diameter portion on the direction X2 side, and the end portion 51B is inserted into the button 510. The spring 511 is disposed around the small-diameter portion of the button 510, and one end of the spring 511 is fixed to the large-diameter portion of the button 510.

In a non-operating state of the valve mechanism 454 shown in FIG. 20, the inside of the first storage member 522 and the inside of the second storage member 521 are liquid-tightly sealed by the O-ring 55. In this state, the inside of the second connection pipe line 521A communicates with the outside via the hole portion 51A and a gap between the enlarged-diameter end portion of the second storage member 521 and the tubular body 512. Therefore, in a case where the suction device is operated, air can be suctioned from the enlarged-diameter end portion of the second storage member 521. On the other hand, in the operating state of the valve mechanism 454 shown in FIG. 21, the sealing property inside the storage member 52 is maintained by the O-ring 54.

As shown in FIG. 21, in the piston 51, the hole portion 52A is provided at a position facing the first connection pipe line 522A, and the hole portion 51A is provided at a position facing the second connection pipe line 521A. In a state in which the button 510 is pressed, the first connection pipe line 522A and the hole portion 52A face each other, and the second connection pipe line 521A and the hole portion 51A face each other. Therefore, the first connection pipe line 522A and the second connection pipe line 521A communicate with each other via the hole portion 51A and the hole portion 52A. Therefore, by operating the suction device connected to the suction nozzle 455, a negative pressure can be generated in the treatment tool pipe line 451, and blood or an object such as a clip can be suctioned from the treatment tool outlet port 44C. In FIG. 21, a trajectory of the clip to be suctioned is indicated by a one-dot chain line. Since the hole portion 52A and the hole portion 51A are each configured as an elongated hole and are disposed to be shifted in the direction X, even a clip having an elongated shape can be moved smoothly from the operation unit pipe line 450 to the suction nozzle 455.

As shown in FIG. 21, in a case where the piston 51 is at the second position P2, in the direction X, an end edge 51Ae of the hole portion 51A on the direction X2 side may be at the same position as an end edge of the first connection pipe line 522A on the direction X1 side or may be on the direction X2 side with respect to the end edge of the first connection pipe line 522A on the direction X1 side. In this way, a path connecting the first connection pipe line 522A and the second connection pipe line 521A can be widened, and a large object can be suctioned.

It is preferable that the inner diameters of the treatment tool pipe line 451, the branch member 452, the operation unit pipe line 450, the first connection pipe line 522A, the second storage member 521, the second connection pipe line 521A, and the suction nozzle 455 are the same. For example, by setting the inner diameter to 6 mm or more, the clip can be suctioned. In this way, since there is no portion that is narrowed in the pipe line extending from the treatment tool pipe line 451 to the suction nozzle 455, a pressure loss during suction is less likely to occur, and the suction force is maintained.

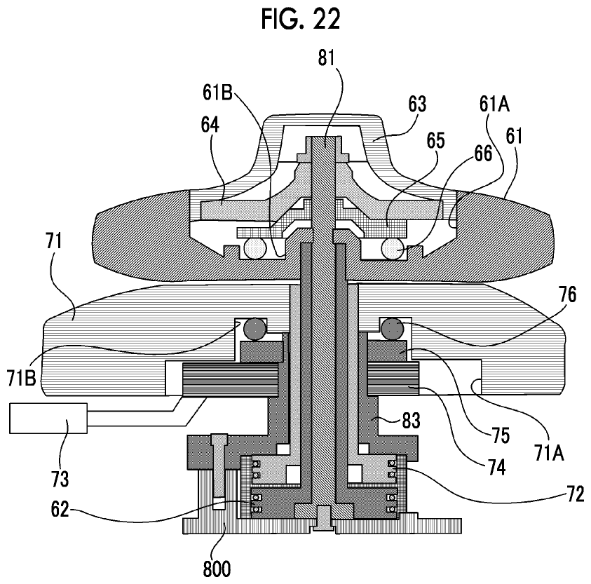

FIG. 22 is a schematic cross-sectional view of a bending operation unit that performs a bending operation of the bendable part 43 disposed in the operation unit 45. JP2011-30866A discloses an endoscope including an insertion tube to be inserted into a body cavity, an operation unit that is consecutively installed at a proximal end of the insertion tube, an angle knob that is provided in the operation unit and that is rotated in a case of bending a distal end portion of the insertion tube, and a lock mechanism that locks the angle knob to prevent rotation.

The operation unit 45 is provided with an LR knob 61 for performing a left-right bending operation and a UD knob 71 for performing an up-down bending operation, as first operation members that are rotatably operable and connected to the bendable part 43. A base portion 800 is fixed to the housing 45A of the operation unit 45, and a rotary shaft 81 is supported by the base portion 800. An LR pulley 62 is provided on an outer periphery of the rotary shaft 81 to be rotatable around the rotary shaft 81. The LR knob 61 is provided on an outer periphery of the LR pulley 62 and is constituted by a disk-shaped member fixed to the LR pulley 62.

A UD pulley 72 is provided on an outer periphery of the LR pulley 62 to be rotatable around the rotary shaft 81. The UD knob 71 is provided on an outer periphery of the UD pulley 72 and is constituted by a disk-shaped member fixed to the UD pulley 72.

Operation wires connected to the first support member 13 of the bendable part 43 are fixed to the LR pulley 62 and the UD pulley 72, respectively. In a case where the LR knob 61 and the LR pulley 62 are rotated, the operation wire is pulled. As a result, the bendable part 43 is bent to the left and right. In a case where the UD knob 71 and the UD pulley 72 are rotated, the operation wire is pulled. As a result, the bendable part 43 is bent up and down.

A recessed portion 61A is provided in the LR knob 61. An annular groove 61B surrounding the rotary shaft 81 is formed in a bottom surface of the recessed portion 61A, and a friction force generating member 66 capable of generating a frictional force with the LR knob 61 is disposed in the groove 61B. The friction force generating member 66 includes, for example, an O-ring or a C-ring made of an elastic material.

In the recessed portion 61A, a push ring 65 is further disposed above the friction force generating member 66, and a brake ring 64 is disposed above the push ring 65. The recessed portion 61A is closed by an LR lock knob 63 having a hat shape.

The brake ring 64 is provided on an outer periphery of the rotary shaft 81 to be rotatable around the rotary shaft 81. The LR lock knob 63 and the brake ring 64 are fixed to each other and rotate integrally. The push ring 65 is provided on the outer periphery of the rotary shaft 81 to be rotatable around the rotary shaft 81 and movable along the rotary shaft 81. A spiral inclined surface that abuts on a projection portion provided on the brake ring 64 is provided on a surface of the push ring 65 on a brake ring 64 side. In a case where the LR lock knob 63 and the brake ring 64 are rotated in one direction, the projection portion of the brake ring 64 moves along the inclined surface of the push ring 65, so that the push ring 65 moves in a direction of the friction force generating member 66. With this movement, the push ring 65 presses the friction force generating member 66 toward the LR knob 61, a large frictional force is generated on the LR knob 61, the rotation of the LR knob 61 is prevented, and the LR knob 61 is locked. In a case where the LR lock knob 63 and the brake ring 64 are rotated in a direction opposite to the one direction from a state in which the LR knob 61 is locked, the frictional force between the friction force generating member 66 and the LR knob 61 is reduced, and the LR knob 61 is rotatable.

A recessed portion 71A is provided in the UD knob 71. An annular groove 71B surrounding the rotary shaft 81 is formed in a bottom surface of the recessed portion 71A, and a friction force generating member 76 capable of generating a frictional force with the UD knob 71 is disposed in the groove 71B. The friction force generating member 76 includes, for example, an O-ring or a C-ring made of an elastic material.

In the recessed portion 71A, a push ring 75 is further disposed below the friction force generating member 76, and a brake ring 74 is disposed below the push ring 75. The UD lock knob 73 is fixed to the brake ring 74.

The brake ring 74 is provided on an outer periphery of the rotary shaft 81 to be rotatable around the rotary shaft 81. The push ring 75 is provided on the outer periphery of the rotary shaft 81 to be rotatable around the rotary shaft 81 and movable along the rotary shaft 81. A spiral inclined surface that abuts on a projection portion provided on the brake ring 74 is provided on a surface of the push ring 75 on a brake ring 74 side. In a case where the UD lock knob 73 and the brake ring 74 are rotated in one direction, the projection portion of the brake ring 74 moves along the inclined surface of the push ring 75, so that the push ring 75 moves in a direction of the friction force generating member 76. With this movement, the push ring 75 presses the friction force generating member 76 toward the UD knob 71, a large frictional force is generated on the UD knob 71, the rotation of the UD knob 71 is prevented, and the UD knob 71 is locked. In a case where the UD lock knob 73 and the brake ring 74 are rotated in a direction opposite to the one direction from a state in which the UD knob 71 is locked, the frictional force between the friction force generating member 76 and the UD knob 71 is reduced, and the UD knob 71 can be rotated.

The LR lock knob 63 and the brake ring 64 constitute a second operation member. The UD lock knob 73 and the brake ring 74 constitute a second operation member. The push ring 65 and the push ring 75 each constitute a movable member or a pressing body. The groove 61B constitutes an accommodation portion that accommodates the friction force generating member 66. The groove 71B constitutes an accommodation portion that accommodates the friction force generating member 76. The friction force generating member 66 and the push ring 65 constitute a restriction mechanism that restricts the rotation of the LR knob 61. The friction force generating member 76 and the push ring 75 constitute a restriction mechanism that restricts the rotation of the UD knob 71.

The above-described brake mechanism is different from a general endoscope device described in JP2021-137183A, the general endoscope device including an insertion part that is inserted into a subject and that includes a bendable movable portion on a distal end side, an operation unit that is provided on a proximal end side of the insertion part and that operates each unit of a device including the bendable movable portion, a bending operation mechanism that causes the bendable movable portion to perform a bending operation in response to a rotational movement operation of a bending operation unit provided on the operation unit, and a bending holding mechanism that causes a pressing portion to abut against or to be spaced apart from a rotational movement surface that rotates together with the bending operation unit in response to a rotational movement operation of a brake operation unit provided in the operation unit together with the bending operation unit, to generate or release a brake force with respect to the rotational movement surface.

In the above description, the single-use endoscope 40 has been described as being for single use, but may be configured to be usable multiple times.

As described so far, at least the following matters are described in the present specification.

(1)

An endoscope comprising:
an insertion part that is inserted into a subject;
a bendable part that is disposed in the insertion part and that is capable of performing a bending operation;
a first distal end portion that is disposed on a distal end side of the bendable part in the insertion part;
an illumination portion that is disposed on a distal end surface of the first distal end portion and that emits illumination light; and
an observation portion that is disposed on the distal end surface of the first distal end portion and on which reflected light obtained by reflection of the illumination light by the subject is incident,
in which the first distal end portion includes a first member in which the observation portion is disposed, a second member that is disposed adjacent to the first member in a circumferential direction around a longitudinal direction of the insertion part, and an adhesion portion that adheres the first member to the second member.
   (2)

The endoscope according to (1),
in which the adhesion portion is disposed inward of an outer peripheral surface of the first distal end portion.
   (3)

The endoscope according to (1) or (2),
in which the adhesion portion includes a region that is reachable by ultraviolet light transmitted through the first member or the second member.
   (4)

The endoscope according to any one of (1) to (3), further comprising:
an insertion opening that is disposed on the distal end surface of the first distal end portion and into which a treatment tool is insertable,
in which the first member and the second member constitute an inner peripheral surface of the insertion opening.
   (5)

The endoscope according to (4), further comprising:
an imaging element that is disposed inside the first member and on which the reflected light incident on the observation portion forms an image.
(6)

The endoscope according to (4),
in which the illumination portion is disposed in the first member, and
the endoscope further comprises a light emitting element that is disposed inside the first member and that supplies illumination light to the illumination portion.
   (7)

The endoscope according to (5),
in which the illumination portion is disposed in the first member,
the endoscope further comprises a light emitting element that is disposed inside the first member and that supplies illumination light to the illumination portion, and
the imaging element and the light emitting element are modularized.
   (8)

The endoscope according to any one of (1) to (7),
in which the adhesion portion includes a first adhesion region and a second adhesion region having different shapes.
   (9)

The endoscope according to (8),
in which the first adhesion region is a region in which the first member and the second member are at least adhered by engagement between the first member and the second member.
   (10)

The endoscope according to any one of (1) to (9), further comprising:
a second distal end portion that is disposed between the first distal end portion and the bendable part in the insertion part and that has an outer diameter greater than an outer diameter of the first distal end portion,
in which proximal end sides of the first member and the second member are disposed inside the second distal end portion.
   (11)

The endoscope according to any one of (1) to (10), further comprising:
an insertion opening that is disposed on the distal end surface of the first distal end portion and into which a treatment tool is insertable,
in which the first distal end portion includes a third member that is disposed between the first member and the second member in the circumferential direction and that is adhered to the first member and the second member, and
the first member, the second member, and the third member constitute an inner peripheral surface of the insertion opening.
   [1]

An endoscope comprising:
an insertion part that is inserted into a subject;
a structure that is disposed in the insertion part and that is capable of performing a bending operation;
a first support member that supports a distal end side of the structure;
a second support member that supports a proximal end side of the structure;
a tubular coating member that is made of a non-stretchable material and that coats the structure, the first support member, and the second support member;
a first adhesion portion that adheres a distal end side of the coating member to the first support member; and
a second adhesion portion that adheres a proximal end side of the coating member to the second support member,
in which, in a longitudinal direction of the insertion part, a length of a first portion of the coating member between the first adhesion portion and the second adhesion portion is greater than a length of the structure.
   [2]

The endoscope according to [1],
in which the length of the first portion is 1.1 times or more and 1.5 times or less the length of the structure.
   [3]

The endoscope according to [1] or [2],
in which the length of the first portion is greater than a length of the structure in a maximally bent state.
   [4]

The endoscope according to any one of [1] to [3],
in which the first portion has slack in a state in which the structure extends linearly.
   [5]

The endoscope according to [4],
in which the first portion has slack in a state in which the structure is bent.
   [6]

The endoscope according to [5],
in which the first portion has slack in a maximally bent state of the structure.
   [7]

The endoscope according to any one of [1] to [6],
in which the length of the first portion is 70 mm or more and 150 mm or less, and
the length of the structure is 50 mm or more and 100 mm or less.
   [8]

The endoscope according to any one of [1] to [7],
in which an outer diameter of the first portion is 8 mm or more and 16 mm or less, and
an outer diameter of the structure is 9 mm or more and 15 mm or less.
   [9]

The endoscope according to any one of [1] to [8],
in which an elastic modulus of the coating member is 18 MPa or less at an elongation ratio of 100%.
   [10]

The endoscope according to any one of [1] to [9],
in which the coating member contains a filler.
   [11]

The endoscope according to [10],
in which the filler has X-ray shielding properties.
   [12]

The endoscope according to [10] or [11],
in which the filler contains barium sulfate.
   [13]

The endoscope according to any one of [10] to [12],
in which a content of the filler is 51% or less.
   {1}

An endoscope comprising:
an insertion part that is inserted into a subject;
an operation unit that is disposed on a proximal end side of the insertion part and that is configured to operate the insertion part;
an insertion opening for a treatment tool that is disposed at a distal end of the insertion part;
a pipe line that is disposed in the operation unit and that communicates with the insertion opening;
a suction nozzle that is disposed in the operation unit and that is connectable to a suction device;
an operation member that is disposed in the operation unit, that is movable, and that has an internal space, the operation member performing an operation of causing the pipe line and the suction nozzle to communicate with each other;
a storage member that forms a storage space for movably storing the operation member and that has a first connection pipe line connecting the pipe line and the storage space to each other and a second connection pipe line connecting the suction nozzle and the storage space to each other; and
a hole portion that is provided in the operation member, that causes the first connection pipe line and the second connection pipe line to communicate with each other in a case where the operation member is at a second position in the storage space, and that does not cause the first connection pipe line and the second connection pipe line to communicate with each other in a case where the operation member is at a first position in the storage space.
   {2}

The endoscope according to {1},
in which the storage member is constituted by connecting a first storage member having a bottomed tubular shape and including the first connection pipe line and a second storage member having a tubular shape and including the second connection pipe line.
   {3}

The endoscope according to {1} or {2},
in which the hole portion of the operation member includes a first opening that is provided at a position capable of facing the first connection pipe line and a second opening that is provided at a position where the second opening is capable of facing the second connection pipe line, and
the first opening and the second opening are at different positions in a movable direction of the operation member.
   {4}

The endoscope according to {3},
in which the first opening and the second opening are each formed as an elongated hole extending in the movable direction of the operation member.
   {5}

The endoscope according to {3} or {4},
in which the operation member has a protruding portion that protrudes from a housing of the operation unit,
the second connection pipe line is disposed at a position closer to the protruding portion than the first connection pipe line in the movable direction of the operation member, and
the second opening is disposed at a position closer to the protruding portion than the first opening in the movable direction of the operation member.
   {6}

The endoscope according to {5},
in which, in a case where the operation member is at the second position, an end edge of the second opening on a first opening side is located at the same position as an end edge of the first connection pipe line on a second connection pipe line side or is located on a side opposite to the second connection pipe line side with respect to the end edge of the first connection pipe line on the second connection pipe line side, in the movable direction of the operation member.
   {7}

The endoscope according to {5} or {6},
in which the storage member is constituted by connecting a first storage member having a bottomed tubular shape and including the first connection pipe line and a second storage member having a tubular shape and including the second connection pipe line,
the first storage member has a portion that is located on a side opposite to the second connection pipe line side with respect to the first connection pipe line in the movable direction of the operation member, and
an end portion of the operation member is configured to be stored in the portion of the first storage member in a case where the operation member is at the second position.
   {8}

The endoscope according to {7}, further comprising:
a sealing member that is provided on an outer periphery of the end portion of the operation member.
   <1>

An endoscope comprising:
an insertion part that is inserted into a subject;
a bendable part that is disposed in the insertion part and that is capable of performing a bending operation;
a first distal end portion that is disposed on a distal end side of the bendable part in the insertion part;
an illumination portion that is disposed on a distal end surface of the first distal end portion and that emits illumination light; and
an observation portion that is disposed on the distal end surface of the first distal end portion and on which reflected light obtained by reflection of the illumination light by the subject is incident,
in which the first distal end portion includes a plurality of members that are arranged and adhered in a circumferential direction around a longitudinal direction of the insertion part, and a restricting structure that is provided in the plurality of members and that restricts relative movement of the plurality of members in the longitudinal direction.
   <2>

The endoscope according to <1>, further comprising:
an annular second distal end portion that is disposed between the first distal end portion and the bendable part in the insertion part and that has an outer diameter greater than an outer diameter of the first distal end portion,
in which proximal end sides of the plurality of members are disposed inside the second distal end portion.
   <3>

The endoscope according to <2>, further comprising:
an extending portion that is provided to extend from at least one of the plurality of members to a proximal end side and that penetrates the second distal end portion;
a tubular member that is provided in the bendable part and into which the extending portion is inserted;
a through-hole that is provided in a region of the tubular member facing the extending portion and that penetrates the tubular member in a radial direction;
a hole portion that is provided at a position facing the through-hole in the extending portion; and
an adhesive that fills the hole portion and the through-hole.
   <4>

The endoscope according to any one of <1>to <3>,
in which the plurality of members have butted surfaces that are butted against each other, and
the butted surfaces are configured such that ultraviolet light transmitted through at least one of the plurality of members reaches the butted surfaces.
   <5>

The endoscope according to any one of <1>to <4>, further comprising:
an insertion opening that is disposed on the distal end surface of the first distal end portion and into which a treatment tool is insertable,
in which the plurality of members constitute an inner peripheral surface of the insertion opening.
   <6>

An endoscope comprising:
an insertion part that is inserted into a subject;
a bendable part that is disposed in the insertion part and that is capable of performing a bending operation;
a first distal end portion that is disposed on a distal end side of the bendable part in the insertion part;
an illumination portion that is disposed on a distal end surface of the first distal end portion and that emits illumination light; and
an observation portion that is disposed on the distal end surface of the first distal end portion and on which reflected light obtained by reflection of the illumination light by the subject is incident,
in which the first distal end portion includes a plurality of members that are arranged in a circumferential direction around a longitudinal direction of the insertion part, and
at least one of the plurality of members has a transmittance of 50% or more with respect to a wavelength of 250 nm or more and 450 nm or less.
   <7>

An endoscope comprising:
an insertion part that is inserted into a subject;
a bendable part that is disposed in the insertion part and that is capable of performing a bending operation;
a first distal end portion that is disposed on a distal end side of the bendable part in the insertion part;
an illumination portion that is disposed on a distal end surface of the first distal end portion and that emits illumination light;
an observation portion that is disposed on the distal end surface of the first distal end portion and on which reflected light obtained by reflection of the illumination light by the subject is incident; and
an insertion opening that is disposed on the distal end surface of the first distal end portion and into which a treatment tool is insertable,
in which the first distal end portion includes a plurality of members arranged in a circumferential direction around a longitudinal direction of the insertion part, and
the plurality of members constitute an inner peripheral surface of the insertion opening.
   [1]

An endoscope comprising:
an insertion part that is inserted into a subject;
a bendable part that is disposed in the insertion part and that is capable of performing a bending operation;
an operation unit that is disposed on a proximal end side of the insertion part and that is capable of performing a bending operation of the bendable part;
a first operation member that is provided in the operation unit and that is connected to the bendable part and that is capable of performing a rotating operation;
a friction force generating member that is provided adjacent to the first operation member in the operation unit and that is capable of generating a frictional force between the first operation member and the friction force generating member;
a second operation member that is provided in the operation unit and that is operable; and
a movable member that is provided between the second operation member and the friction force generating member, that moves in response to an operation of the second operation member, and that is configured to press the friction force generating member against the first operation member.
   [2]

The endoscope according to [1], further comprising:
an accommodation portion that accommodates the friction force generating member provided in the first operation member.
   [3]

The endoscope according to [1] or [2],
in which the friction force generating member is a C-ring or an O-ring made of an elastic material.
   [4]

The endoscope according to any one of [1] to [3],
in which the second operation member is configured to rotate about a rotary shaft of the first operation member, and
the movable member is configured to move in a direction of the rotary shaft in response to rotation of the second operation member.
   [5]

An endoscope comprising:
an insertion part that is inserted into a subject;
a bendable part that is disposed in the insertion part and that is capable of performing a bending operation;
an operation unit that is disposed on a proximal end side of the insertion part and that is capable of performing a bending operation of the bendable part;
a first operation member that is provided in the operation unit and that is connected to the bendable part and that is capable of performing a rotating operation; and
a restriction mechanism that is provided in the operation unit and that restricts rotation of the first operation member,
in which the restriction mechanism includes a friction force generating member that generates a frictional force between the first operation member and the friction force generating member, and a pressing body that presses the friction force generating member against the first operation member.
   [6]

The endoscope according to [5], further comprising:
a second operation member that is provided in the operation unit and that is operable,
in which the pressing body is configured to move in response to an operation of the second operation member.
   [7]

The endoscope according to [6],
in which the second operation member is configured to rotate about a rotary shaft of the first operation member, and
the pressing body is configured to move in a direction of the rotary shaft in response to rotation of the second operation member.

### EXPLANATION OF REFERENCES

11: first distal end portion
12: second distal end portion
13: first support member
13A, 14A: outer peripheral surface
13K, 111A, 112A, 113A: through-hole
14: second support member
41: insertion part
40: single-use endoscope
42: flexible part
43: bendable part
44: distal end portion
44A: observation portion
44B: illumination portion
44C: treatment tool outlet port
44D: air/water supply port
44E: auxiliary water supply port
44N: air/water supply nozzle
45: operation unit
45A: housing
46: first cord part
46A, 47A: connector
47: second cord part
50: adapter
51: piston
51A, 52A, 112K: hole portion
51B: end portion
51Ae: end edge
52: storage member
54, 55: O-ring
61: LR knob
61A, 71A, 111bo, 111co, 113bo, 114, 115, 116, 123A: recessed portion
61B, 71B: groove
62: LR pulley
64, 74: brake ring
65, 75: push ring
66, 76: friction force generating member
71: UD knob
72: UD pulley
80: control device
80A: connector connection portion
80B: input device
81: rotary shaft
90: display device
100: endoscope device
111: upper member
111i, 112i, 113i: curved surface
111b, 111Sa, 111c, 111Sb, 112b, 112Sa, 112c, 112Sb, 113b, 113Sa, 113c, 113Sb: end surface
112: right-side member
112B: extending portion
112ct, 112bt, 113ct: projection portion
113: left-side member
123, 800: base portion
124, 129: protruding portion
125: first through-hole
125A, 127A: opening
126: second through-hole
127: third through-hole
128: fourth through-hole
200: tablet terminal
430: structure
430L, 432L: length
431: bending piece
432: coating member
432C: first portion
440: observation module
441: optical system
442: imaging element
443: light emitting unit
444: light shielding case
445: resin cover
446: cable
450: operation unit pipe line
451: treatment tool pipe line
452: branch member
453: treatment tool inlet port
454, 456: valve mechanism
455: suction nozzle
461: air/water supply pipe line
461A: air/water supply connection fitting
462: water supply pipe line
462A: auxiliary water supply fitting
471: signal line
510: button
511: spring
512: tubular body
512G: guide groove
521: second storage member
521A: second connection pipe line
521T: engaging projection portion
522: first storage member
522A: first connection pipe line
522B: bottom portion
CP1, CP2, CP3: center

What is claimed is:

1. An endoscope comprising:
an insertion part that is inserted into a subject;
a bendable part that is disposed in the insertion part and that is capable of being bent;
a first distal end portion that is disposed on a distal end side of the bendable part in the insertion part;
an illumination portion that is disposed on a distal end surface of the first distal end portion and that emits illumination light; and
an observation portion that is disposed on the distal end surface of the first distal end portion and on which reflected light obtained by reflection of the illumination light by the subject is incident,
wherein the first distal end portion includes a first member in which the observation portion is disposed, a second member that is disposed adjacent to the first member in a circumferential direction around a longitudinal direction of the insertion part, and an adhesion portion that adheres the first member to the second member.

2. The endoscope according to claim 1,
wherein the adhesion portion is disposed inward of an outer peripheral surface of the first distal end portion.

3. The endoscope according to claim 1,
wherein the adhesion portion includes a region that is reachable by ultraviolet light transmitted through the first member or the second member.

4. The endoscope according to claim 1, further comprising:
an insertion opening that is disposed on the distal end surface of the first distal end portion and into which a treatment tool is insertable,
wherein the first member and the second member define an inner peripheral surface of the insertion opening.

5. The endoscope according to claim 2, further comprising:
an insertion opening that is disposed on the distal end surface of the first distal end portion and into which a treatment tool is insertable,
wherein the first member and the second member define an inner peripheral surface of the insertion opening.

6. The endoscope according to claim 3, further comprising:
an insertion opening that is disposed on the distal end surface of the first distal end portion and into which a treatment tool is insertable,
wherein the first member and the second member define an inner peripheral surface of the insertion opening.

7. The endoscope according to claim 4, further comprising:
an imaging element that is disposed inside the first member and on which the reflected light incident on the observation portion forms an image.

8. The endoscope according to claim 5, further comprising:
an imaging element that is disposed inside the first member and on which the reflected light incident on the observation portion forms an image.

9. The endoscope according to claim 6, further comprising:
an imaging element that is disposed inside the first member and on which the reflected light incident on the observation portion forms an image.

10. The endoscope according to claim 4,
wherein the illumination portion is disposed in the first member, and
the endoscope further comprises a light emitting element that is disposed inside the first member and that supplies illumination light to the illumination portion.

11. The endoscope according to claim 5,
wherein the illumination portion is disposed in the first member, and
the endoscope further comprises a light emitting element that is disposed inside the first member and that supplies illumination light to the illumination portion.

12. The endoscope according to claim 6,
wherein the illumination portion is disposed in the first member, and
the endoscope further comprises a light emitting element that is disposed inside the first member and that supplies illumination light to the illumination portion.

13. The endoscope according to claim 7,
wherein the illumination portion is disposed in the first member,
the endoscope further comprises a light emitting element that is disposed inside the first member and that supplies illumination light to the illumination portion, and
the imaging element and the light emitting element are modularized.

14. The endoscope according to claim 8,
wherein the illumination portion is disposed in the first member,
the endoscope further comprises a light emitting element that is disposed inside the first member and that supplies illumination light to the illumination portion, and
the imaging element and the light emitting element are modularized.

15. The endoscope according to claim 9,
wherein the illumination portion is disposed in the first member,
the endoscope further comprises a light emitting element that is disposed inside the first member and that supplies illumination light to the illumination portion, and
the imaging element and the light emitting element are modularized.

16. The endoscope according to claim 1,
wherein the adhesion portion includes a first adhesion region and a second adhesion region having different shapes.

17. The endoscope according to claim 16,
wherein the first adhesion region is a region in which the first member and the second member are engaged with each other.

18. The endoscope according to claim 1, further comprising:
a second distal end portion that is disposed between the first distal end portion and the bendable part in the insertion part and that has an outer diameter greater than an outer diameter of the first distal end portion,
wherein proximal end sides of the first member and the second member are disposed inside the second distal end portion.

19. The endoscope according to claim 1, further comprising:
an insertion opening that is disposed on the distal end surface of the first distal end portion and into which a treatment tool is insertable,
wherein the first distal end portion includes a third member that is disposed between the first member and the second member in the circumferential direction and that is adhered to the first member and the second member, and
the first member, the second member, and the third member constitute an inner peripheral surface of the insertion opening.
